(19) United States
(12) Patent Application Publication
(10) Pub. No.: US 2026/0077045 A1
(43) Pub. Date: Mar. 19, 2026

(54) METHOD TO GENERATE IMPROVING CAR-T CELLS

(71) Applicants: INSTITUT NATIONAL DE LA SANTÉ ET DE LA RECHERCHE MÉDICALE, Paris (FR); UNIVERSITÉ PARIS CITÉ, Paris (FR); CENTRE NATIONAL DE LA RECHERCHE SCIENTIFIQUE, Paris (FR)

(72) Inventors: Frédéric PENDINO, Paris (FR); Emmanuel DONNADIEU, Paris (FR); Mattia FUMAGALLI, Paris (FR); Dongjie AN, Paris (FR)

(21) Appl. No.: 19/109,674

(22) PCT Filed: Oct. 6, 2023
(86) PCT No.: PCT/EP2023/077788

§ 371 Date: Mar. 7, 2025

(30) Foreign Application Priority Data
Oct. 7, 2022 (EP) 22306511.1

(51) Int. Cl. A61K 40/11; A61K 40/31; A61K 40/42; A61P 35/00; C07K 14/725; C07K 16/28; C12N 9/22; C12N 15/11; C12N 15/86

(52) U.S. Cl. CPC A61K 40/11; A61K 40/31; A61K 40/4204; A61K 40/4211; A61P 35/00; C07K 14/7051; C07K 16/2803; C07K 16/2863; C12N 9/226; C12N 15/11; C12N 15/86; C12N 2310/20

(57) ABSTRACT

The present invention relates to the adoptive therapy using notably CAR-T cells. Here the inventors used a lentiviral vector approach to silence RINF expression in a shRNA-dependent manner and evaluate the consequences of RINF silencing on human CAR-T cells proliferation ex vivo and their functionality and capacity to eradicate tumor cells in vivo. More, the proposed methodology to improve CAR-T cells persistence and efficacy by disrupting RINF/CXXC5 is not restricted to patients suffering from hematological or solid cancers (anti-CD19, anti-EGFR, anti-BCMA . . . ) but could be also used to improve the efficacy of ACT in non-cancer diseases by such as lupus (1), cardiac fibrosis (2) or aging related-disorders (3). Thus, the present invention relates to an immune cell characterized in that it is defective for RINF.

### FIELD OF THE INVENTION

The present invention relates to an immune cell characterized in that it is defective for RINF.

### BACKGROUND OF THE INVENTION

Cancer immunotherapies are increasingly used in the treatment of cancers and unprecedent clinical results were obtained in several malignancies. Among the novel strategies that have been developed, two approaches that target T cells, key players in the fight against cancer, appear particularly efficient, namely immune checkpoint inhibitors (such as anti-PD-1 or anti-CTLA4 antibodies) and chimeric antigen receptor (CAR) T-cells. In the latter approach, T cells are isolated from cancer patients and are engineered to express an activating fusion receptor that can recognize a cell surface target antigen that is found on tumor cells (1). The binding of the CAR to its target triggers activation signals that permit cytotoxic T cells to kill malignant cells. Within the past few years, clinical trials using T cells expressing CARs that recognize malignant B cells (such as anti-CD19 and anti-CD20 CARs) have shown high rates of response in B-cell malignancies that are refractory to chemotherapy (2). Nonetheless, complete remission is not observed in all these patients (but only in ˜50-70% of them), is restricted to certain leukemia subtypes (B-cells leukemia/lymphoma), and this therapeutic approach is so far disappointing in most of solid malignancies (3, 4). It is thus crucial to improve CAR T cells efficiency. Among the main reasons that are limiting their clinical efficacy, the lack of in vivo persistence of CAR-T cells is a major limitation that must be urgently resolved. Lately, several groups have proposed to reprogram T-cells by targeting some epigenetic factors. (5, 6) Unfortunately, these genes are also well-known tumor suppressor genes that are frequently mutated in malignant hemopathies (6, 7) and their gene invalidation in mouse leads to high rates of transformation (8, 9). Thus, identifying novel epigenetic factors whose gene silencing (or invalidation) would improve long-term T-cells persistence and efficacy without increasing the risk of transformation into T cells lymphoma/leukemia, would be an important step to develop safer strategies for next generations of ACT.

In 2009, the team of the inventors has identified the CXXC5 gene encoding a Retinoid-Inducible Nuclear Factor (RINF) and demonstrated its role in human hematopoiesis (10). Indeed, the group has first demonstrated its role during terminal granulocytic differentiation in a model of acute promyelocytic leukemia. (10) Lately, they have demonstrated its role during erythropoiesis and shown that shRNA-mediated RINF knockdown affects human erythropoiesis and mitigates red blood cells production (11). The last few years, other groups using mouse models, have also suggested the involvement of Cxxc5/Rinf during the maturation of various cell types such as neural stem cells (12), endothelial cells (13), myoblasts and myofibroblasts (14), kidney (15), cutaneous wound healing and bone maintenance (16, 17), and hair-regrowth (18). Of note, the number of lymphoid cells (including T cells) was not altered in Rinf −/− KO models, suggesting the absence of function of this factor in human T-cell proliferation (19).

The inventors have also been pioneered in studying RINF in cancer and leukemia. If the coding sequence of this gene is not frequently mutated in malignant hemopathies (20, 21), they have shown that RINF expression is heterogeneous and upregulated in several solid tumors such as malignant melanoma, thyroid, breast cancer (22). Strikingly, high RINF mRNA expression is an unfavorable prognostic factor in breast cancer (22-24) as well as in acute myeloid leukemia (AML) (25-27). Statistical multivariate analyses have demonstrated that high RINF mRNA expression (observed in approximately one third of the patients) is an independent marker of known risk factors in AML (such as of cytogenetics and mutations of FLT3-ITD, NPM1 and CEBPA) (27). Recently, RINF expression was shown to contribute to prostate cancer resistance to anti-androgens in a cell-intrinsic dependent manner (28).

At the molecular level, the exact mode of action of RINF remains a topic of debate and probably depends on the cellular context. It has been suggested that RINF would inhibit the WNT-β-catenin signaling pathway (12, 15-18, 27, 29), through a cytoplasmic interaction with Dishevelled proteins DVL and DVL2 (12, 16, 30). However, in most of the studies, the subcellular localization of RINF protein is mainly or exclusively nuclear and RINF contains a Nuclear Localization Signal (10) that has been functionally validated (31). Moreover, RINF strongly associates with chromatin (10), probably through its conserved zinc-finger domain (CXXC) that plays a central role and provides the capacity to bind CpG islands (32). This CXXC-domain is almost identical to the one harbored by TET1 and TET3, two epigenetic modulators involved in the erasure of DNA-methylation marks together with TET2 (that lacks this CXXC-domain). Thus, RINF could interfere with TET-activities, hydroxymethylation, and gene transcription, even though these data are inconsistent even in the same model (19, 33, 34). Other studies suggest a role as a transcriptional regulator (13, 35, 36). Since the RINF protein does not contain any known trans-activation or trans-repression domain, it was suggested that it could act as a cofactor of transcription (32). Consistent with the later hypothesis, this nuclear factor has been shown to interact with established transcription factors such as Vitamin-D3-Receptor (VDR) (37), FOXL2 (38), and SMAD3/4 proteins (39). Another study suggests that RINF would be a binding partner of ATM and would mediate DNA-damage induced-activation of TP53 (34).

### SUMMARY OF THE INVENTION

Here the inventors used a lentiviral vector approach to silence RINF expression in a shRNA-dependent manner and evaluate the consequences of RINF silencing on human CAR-T cells proliferation ex vivo and their functionality and capacity to eradicate tumor cells in vivo. More, the proposed methodology to improve CAR-T cells persistence and efficacy by disrupting RINF/CXXC5 is not restricted to patients suffering from hematological or solid cancers (anti-CD19, anti-EGFR, anti-BCMA . . . ) but could be also used to improve the efficacy of ACT in non-cancer diseases by such as lupus (40), cardiac fibrosis (41) or aging related-disorders (42).

Thus, the present invention relates to an immune cell characterized in that it is defective for RINF. Particularly, the invention is defined by its claims.

### DETAILED DESCRIPTION OF THE INVENTION

### Immune Cell of the Invention

The inventors showed that inhibition (or knockdown or deletion) of RINF leads to an increased number of T cells, improves CAR T cells expansion and their persistence and efficacity in the treatment of tumor.

A First aspect of the invention relates to an immune cell characterized in that is defective for RINF.

In one embodiment, the gene coding for RINF is deleted or silenced.

In another embodiment, the gene coding for RINF is mutated resulting on a non-viable RNA.

According to the invention plural and singular can be used interchangeably.

As used herein the term “defective for RINF” refers to the inhibition, or blockade of RINF activity and/or expression in the immune cell according to the invention.

In one embodiment, the present invention relates to an immune cell characterized in that it does not express or express reduced levels of RINF.

As used herein, the term “RINF” for “Retinoid-Inducible Nuclear Factor” also known as CXXC5 refers to a transcription factor (or cofactor) in the nucleus of cells, and is involved in myelopoiesis, endothelial differentiation, vessel formation, and oligodendrocyte differentiation. RINF is also described as a negative feedback regulator of the Wnt/0-catenin signaling pathway functioning by direct interaction with the Dishevelled (Dvl) protein in the cytosol. The Uniprot reference for Homo sapiens RINF protein is Q7LFL8 and the Entrez reference for Homos sapiens RINF gene is 51523.

As used herein, the terms “expresses reduced levels of RINF” means that the immune cell expresses less RINF compared to its wild type unmanipulated counterpart.

The term “gene” refers to a natural or synthetic polynucleotide containing at least one open reading frame that is capable of encoding a particular polypeptide or protein after being transcribed or translated.

As used herein the term “deleted” means a total or partial deletion of the gene. A partial deletion can involve the removal of any amount of DNA from the target gene, from 1 base pair (bp) up to almost the entire polypeptide coding region of the gene. A total deletion involves the removal of the entire coding region of the gene with or without flanking sequences, which may or may not include regulatory elements that are required for gene function, for example transcriptional promoters. Furthermore, the deletion may result in the removal of just a regulatory region, such as a promoter, leaving the coding region intact. The result is that no mRNA can be produced and so the gene is rendered defective.

As used herein, the term “mutated gene” as used herein means a gene in which a mutation has occurred. The term “mutation” as used herein means a change in the sequence of a nucleic acid and includes a base substitution, insertion, deletion, inversion, duplication, translocation, and the like used in genetics and the like. The region of the mutation in a mutated gene is not limited to a transcriptional region but includes a regulatory region such as a promoter which is required for gene expression.

As used herein, the term “non-viable RNA” relates to a RNA which is not translated into protein.

As used herein, the terms “inhibition of RINF activity” refers to a decrease of RINF activity of at least 30%, 40%, 50%, 60%, 70%, 80%, 90%, 95% or 99% or more as compared to the activity or level of the RINF protein which is not inhibited. Particularly, the inhibition of RINF activity leads to the absence in the cell of substantial detectable activity of RINF.

Inhibition of RINF activity can also be achieved through repression of RINF gene expression or though RINF gene disruption. According to the invention, said repression reduces expression of RINF in the immune cell of the invention by at least 50, 60, 70, 80, 90, or 95% as to the same cell in the absence of the repression. Gene disruption may also lead to a reduced expression of the RINF protein or to the expression of a non-functional RINF protein. By “non-functional” RINF protein it is herein intended a protein with a reduced activity or a lack of detectable activity as described above.

As used herein, “repression” of gene expression refers to the elimination or reduction of expression of one or more gene products encoded by the subject gene in a cell, compared to the level of expression of the gene product in the absence of the repression. Exemplary gene products include mRNA and protein products encoded by the gene. Repression in some cases is transient or reversible and in other cases is permanent. Repression in some cases is of a functional or full-length protein or mRNA, despite the fact that a truncated or non-functional product may be produced. In some embodiments herein, gene activity or function, as opposed to expression, is repressed. Gene repression is generally induced by artificial methods, i.e., by addition or introduction of a compound, molecule, complex, or composition, and/or by disruption of nucleic acid of or associated with the gene, such as at the DNA level. Exemplary methods for gene repression include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination.

As used herein, a “disruption” of a gene refers to a change in the sequence of the gene, at the DNA level. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

As used herein, the term “immune cell” denotes cell of the innate or adaptive immunity and for example myeloid or lymphoid cells, including lymphocytes, typically T cells and/or NK cells.

As used herein, the term T cells denotes for example CD3+ T cells, CD4+ T cells, CD8+ T cells, TILs T cells (Tumor-infiltrating lymphocytes T cells), NK T cells, Stem cell-like memory T cells (TCSM) or Memory T cells (TCM).

The immune cell can be isolated from blood, bone marrow, lymph, lymphoid organs (notably the thymus) peripheral blood lymphocytes (PBL), peripheral blood mononuclear cells (PBMC) or from a biopsy when these cells are for example TILs.

In a particular embodiment, the immune cell are eukaryotic immune cells, such as mammalian cells or human immune cells.

In a particular embodiment, the immune cell of the invention is a lymphocyte including T cell, B cell or NK cell.

In a particular embodiment, the T cell of the invention is a CAR-T cell or T cells armed with recombinant T Cell Receptor (TCR).

In a particular embodiment, the CAR-T cells and all immune cells of the invention are allogenic cells.

Another object of the present invention relates to a population of immune cells of the invention.

As used herein, the term “population” refers to a population of cells, wherein the majority (e.g., at least about 50%, preferably at least about 60%, more preferably at least about 70%, even more preferably at least about 80% and even more preferably at least about 90%) of the total number of cells have the specified characteristics of the cells of interest and express the markers of interest.

In another particular embodiment, the immune cells of the invention is also characterized in that it is defective for SUV39H1, TET1, TET2, TET3 or a combination thereof (see for example WO2018234370 for SUV39H1 or WO2017049166 for the TET family).

### Methods to Obtain the Cells of the Invention

Another object of the invention relates to an ex vivo or in vitro method to obtain immune cells characterized in that it is defective for RINF comprising a step consisting in inhibiting the expression and/or activity of RINF in the immune cells.

In one embodiment, the invention relates to an ex vivo or in vitro method to obtain immune cells characterized in that it does not express or expresses reduced levels of RINF comprising a step consisting in inhibiting the RINF expression in the immune cells.

Particularly, the method of the invention is particularly useful to obtain engineered immune cells, which are defective for RINF.

Accordingly, the methods of the invention will allow to obtain engineered immune cells, which are defective for RINF.

In one embodiment, the invention relates to an ex vivo or in vitro method to obtain improved immune cells characterized in that it is defective for RINF comprising the following steps:
i) Isolating immune cells from a sample obtained from a subject;
ii) Inhibiting the expression and/or activity of RINF in the immune cells.

In a particular embodiment, the invention relates to an ex vivo or in vitro method to obtain CAR-T cells characterized in that it is defective for RINF comprising the following steps:
i) Isolating immune cells (T cells) from a sample obtained from a subject;
ii) transforming the immune cells (T cells) into CAR-T cells thanks to a known method;
iii) inhibiting the expression and/or activity of RINF in the CAR-T cells obtained in the step ii).

In another particular embodiment, the invention relates to an ex vivo or in vitro method to obtain CAR-T cells characterized in that it is defective for RINF comprising the following steps:
i) Isolating immune cells (T cells) from a sample obtained from a subject;
ii) inhibiting the expression and/or activity of RINF in the immune cells obtained in the step i).
iii) transforming the immune cells (T cells) obtained in the step iii) into CAR-T cells thanks to a known method;

In another particular embodiment, the invention relates to an ex vivo or in vitro method to obtain CAR-T cells characterized in that it is defective for RINF comprising the following steps:
i) Isolating immune cells (T cells) from a sample obtained from a subject;
ii) inhibiting the expression and/or activity of RINF in the immune cells obtained in the step i) and transforming said cells into CAR-T cells thanks to a known method in the same step.

In this case and according to the invention, the inhibition of RINF and the transformation of the cells in CAR-T cells can be done using the same CAR construction (for example the same lentivirus expressing a shRNA anti-RNF and the CAR construction).

In a particular embodiment, the method to obtain CAR-T cells comprise another step of addition of IL-7 and/or IL-15.

Thus, the invention relates to an ex vivo or in vitro method to obtain CAR-T cells characterized in that it is defective for RINF comprising the following steps:
i) Isolating immune cells from a sample obtained from a subject;
ii) add IL-7 and/or IL-15 to the medium;
iii) transforming the immune cells (T cells) into CAR-T cells thanks to a known method;
iv) Inhibiting the expression and/or activity of RINF in the CAR-T cells obtained in the step ii).

An ex vivo or in vitro method to obtain improved immune cells characterized in that it is defective for RINF comprising the following steps:
i. Isolating immune cells from a sample obtained from a subject;
ii. Inhibiting the expression of RINF in the immune cells.

An ex vivo or in vitro method to obtain CAR-T cells characterized in that it is defective for RINF comprising the following steps:
i. Isolating an immune cells from a sample obtained from a subject;
ii. transforming the T cells into CAR-T cells thanks to a known method;
iii. inhibiting the expression of RINF in the CAR-T cells obtained in the step ii).

In a particular embodiment, the IL-7 and/or IL-15 are administrated simultaneously with the RINF inhibitor.

After the end of the protocol and the obtention of the CAR-T cells, these cells are injected to a subject in need thereof.

In another particular embodiment, the invention relates to immune cells characterized in that it is defective for RINF obtainable by the ex vivo or in vitro methods described. In one embodiment, the invention relates to CAR-T cells characterized in that it is defective for RINF obtainable by the ex vivo or in vitro methods described.

As used herein, the term “subject” denotes a mammal, such as a rodent, a feline, a canine, and a primate. Preferably, a subject according to the invention is a human.

As used herein, the term “biological sample” refers to any body fluid or tissue. In one embodiment, the biological sample is blood, bone marrow, lymph, lymphoid organs (notably the thymus) peripheral blood lymphocytes (PBL), peripheral blood mononuclear cells (PBMC) or from a biopsy.

As used herein, “isolating” refers to removal of a cell or a cell population from its natural environment. As used herein, “isolated” refers to a cell or a cell population that is removed from its natural environment (such as the sample according to the invention) and that is isolated, purified or separated, and is at least about 75% free, 80% free, 85% free and preferably about 90%, 95%, 96%, 97%, 98%, 99% free, from other cells with which it is naturally present.

According to the invention, the inhibition of the expression and/or activity of RINF can also be done by modifying genetically the immune cells and particularly the CAR-T cells of the invention in order to silence or inactivate the RINF gene.

As used herein, the term “modifying genetically” refers to the addition, suppression or substitution of at least one nucleic acid in the genetic material of the cell.

As used herein the term “to silence the RINF gene” refers to the total or partial suppression of the RINF gene function. This term means that the gene coding for RINF is deleted from the genome or mutated resulting on a non-viable RNA or not functionally expressed.

According to the method of the present invention, the immune cells of the invention are isolated from the sample. All the techniques known by the skilled man may be used.

### Genetically Engineered Cells

In a particular embodiment, the immune cell of the invention further comprises a genetically engineered antigen receptor that specifically binds a target antigen (TCR). In still a particular embodiment, the genetically engineered antigen receptor is a chimeric antigen receptor (CAR) comprising an extracellular antigen-recognition domain that specifically binds to the target antigen.

In some embodiments, the immune cell comprises one or more nucleic acids introduced via genetic engineering that encode one or more antigen receptors.

Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types.

Among the antigen receptors as per the invention are genetically engineered T cell receptors (TCRs) and components thereof, as well as functional non-TCR antigen receptors, such as chimeric antigen receptors (CAR).

### CAR-T Cells

In a particular embodiment, the immune cell of the invention is a CAR-T cell.

As used herein, the terms “Chimeric antigen receptors (CARs)” refer to artificial T cell receptors, chimeric T cell receptors, or chimeric immunoreceptors, and encompass engineered receptors that graft an artificial specificity onto a particular immune effector cell, i.e. the T cells of the invention.

A CAR typically comprises an ectodomain (extracellular domain) and an endodomain (cytoplasmic domain), joined by a transmembrane domain. The ectodomain, expressed on the surface of the cell, comprises an antigen binding domain or receptor domain and optionally a spacer (or hinge) region linking the antigen binding domain to the transmembrane domain. The transmembrane domain is typically a hydrophobic alpha helix that spans across the lipid bilayer of the cell membrane. The endodomain of the CAR is composed of an intracellular signaling module that induces the cell activation upon antigen binding. The endodomain may include several signaling domains, as explained infra.

### Antigen Binding Domain

The extracellular domain of the CAR comprises an antigen binding domain that specifically binds or recognizes a target antigen.

As used herein, “bind” or “binding” refer to peptides, polypeptides, proteins, fusion proteins and antibodies (including antibody fragments) that recognize and contact an antigen. Preferably, it refers to an antigen-antibody type interaction. By “specifically bind” it is meant that the antigen binding domain of the CAR recognizes a specific antigen but does not substantially recognize or bind other molecules in a given sample. The “specific binding” is dependent upon the presence of a particular structure (e.g., an antigenic determinant or epitope). As used herein, the term “specific binding” means the contact between an antigen binding domain of the CAR and an antigen with a binding affinity of at least 10-6 M. In certain aspects, the antigen binding domain of the CAR binds with affinities of at least about 10-7 M, and preferably 10-8 M, 10-9 M, 10-10 M. The binding affinity can be measured by any method available to the person skilled in the art, in particular by surface plasmon resonance (SPR).

In one embodiment, such antigen binding domain is an antibody, preferably a single chain antibody. Preferably, the antibody is a humanized antibody. Particularly, such antigen binding domain is an antibody fragment selected from fragment antigen binding (Fab) fragments, F(ab′)2 fragments, Fab′ fragments, Fv fragments, recombinant IgG (rlgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antibodies are single-chain antibody fragments comprising a variable heavy chain region and/or a variable light chain region, such as scFv. Particularly, such antigen binding domain is selected from a Fab and a scFv.

In embodiments wherein, the antigen targeting domain is a scFv, the scFv can be derived from the variable heavy chain (VH) and variable light chain (VL) regions of an antigen-specific mAb linked by a flexible linker. The scFv retains the same specificity and a similar affinity as the full antibody from which it is derived. The peptide linker connecting scFv VH and VL domains joins the carboxyl terminus of one variable region domain to the amino terminus of the other variable domain without compromising the fidelity of the VH-VL paring and antigen-binding sites. Peptide linkers can vary from 10 to 30 amino acids in length. In one embodiment, the scFv peptide linker is a Gly/Ser linker and comprises one or more repeats of these amino acids.

The extracellular domain of the CAR may comprise one or more antigen binding domain(s).

In a particular embodiment, the CAR specifically binds to a tumor-associated antigen (TAA). In particular, the CAR specifically binds to any TAA expressed at the surface of a tumor cell, particularly CD19, GD2, EGFR, CD20, CD22, CD33, CD138, CD52, CD30, ROR1, HER2, EpCAM, MUC-1, MUC5AC, BCMA, CD38, SLAMF7/CS1, CD123, ILIRAP, IL-13Ra2, LeY, MUC16, PSMA, more preferably the TAA is CD19, CD20, CD22, CD33, CD138, BCMA, SLAMF7/CS1, IL-13Ra2, HER2, EGFR, CD37, CD327, CD276, CD109, or HLA-G.

In another particular embodiment the TCR or CAR targets an intracellular oncoprotein or an intracellular tumor-associated antigen in particular WT-1, NY-ESO-1, MAGE, PRAME, RAS, mesothelin, c-Met, CEA, CSPG-4, EBNA3C, CA-125 or GPA7. In particular, said intracellular oncoprotein or tumor-associated antigen are processed and expressed on the cell surface as peptides bound to histocompatibility (HLA) molecules.

The terms “tumor-associated antigen”, “TAA”, “tumor antigen” and “cancer cell antigen” are used interchangeably herein. In each case, the terms refer to peptides, proteins, glycoproteins or carbohydrates that are specifically or preferentially expressed by cancer cells.

As used herein, the term “antigen” has its general meaning in the art and generally refers to a substance or fragment thereof that is recognized and selectively bound by an antibody or by a T cell antigen receptor, resulting in induction of an immune response. Antigens according to the invention are typically, although not exclusively, peptides and proteins. Antigens may be natural or synthetic and generally induce an immune response that is specific for that antigen.

As used herein, the term “HLA-A2” has its general meaning in the art and refers to a HLA serotype within the HLA-A ‘A’ serotype group and is encoded by the HLA-A*02 allele group including the HLA-A*02:01, HLA-A*02:02, HLA-A*02:03, HLA-A*02:05, HLA-A*02:06, HLA-A*02:07 and HLA-A*02:11 gene products. HLA-A2 is very common in the Caucasian population (40-50%) and provides an ideal cellular target for the first portion because it will be suitable for use in a high proportion of combinations of HLA-A2+ donors and HLA-A2-recipients.

As used herein the term “antibody” and “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes three (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of an amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein, the terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts.

As used herein the term “human antibody” as used herein, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. Such antibodies are designed to maintain the binding specificity of the non-human antibody from which the binding regions are derived, but to avoid an immune reaction against the non-human antibody. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a KD of about 10-7 M or less, such as about 10-8 M or less, such as about 10-9 M or less, about 10-10 M or less, or about 10-11 M or even less.

### Spacer or Hinge Domain

The CAR optionally comprises a spacer or hinge domain linking the antigen binding domain to the transmembrane domain.

In some embodiments, the CAR comprises a hinge sequence between the antigen binding domain and the transmembrane domain and/or between the transmembrane domain and the cytoplasmic domain. One ordinarily skilled in the art will appreciate that a hinge sequence is a short sequence of amino acids that facilitates flexibility.

In particular, the spacer or hinge domain linking the antigen binding domain to the transmembrane domain is designed to be sufficiently flexible to allow the antigen binding domain to orient in a manner that allows antigen recognition.

The hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge domain includes at least a portion of an IgG1, an IgG2, an IgG3, an IgG4, an IgE, an IgM, or an IgA immunoglobulin Fc region that falls within its CH2 and CH3 domains.

Exemplary hinges include, but are not limited to, a CD8a hinge, a CD28 hinge, IgG1/IgG4 (hinge-Fc part) sequences, IgG4 hinge alone, IgG4 hinge linked to CH2 and CH3 domains, or IgG4 hinge linked to the CH3 domain, those described in Hudecek et al. (2013) Clin. Cancer Res., 19:3153, international patent application publication number WO2014031687, U.S. Pat. No. 8,822,647 or published app. No. US2014/0271635. As hinge domain, the invention relates to all or a part of residues 118 to 178 of CD8a (GenBank Accession No. NP_001759.3), residues 135 to 195 of CD8 (GenBank Accession No. AAA35664), residues 315 to 396 of CD4 (GenBank Accession No. NP_000607.1), or residues 137 to 152 of CD28 (GenBank Accession No. NP_006130.1) can be used. Also, as the spacer domain, a part of a constant region of an antibody H chain or L chain (CHI region or CL region) can be used. Further, the spacer domain may be an artificially synthesized sequence.

In some embodiments, for example, the hinge sequence is derived from a CD8 alpha molecule or a CD28 molecule.

### Transmembrane Domain

The transmembrane domain of the CAR functions to anchor the receptor on the cell surface. The choice of the transmembrane domain may depend on the neighboring spacer and intracellular sequences.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD16, CD22, CD33, CD37, CD64, CD80, CD86, CD134, CD137, CD154, ICOS/CD278, GITR/CD357, NKG2D, and DAP molecules. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. A transmembrane domain is thermodynamically stable in a membrane. It may be a single alpha helix, a transmembrane beta barrel, a beta-helix of gramicidin A, or any other structure.

Optionally, a short oligo- or polypeptide linker, preferably between 2 and 10 amino acids in length may form the linkage between the transmembrane domain and the intracellular signaling domain(s) of the CAR. A glycine-serine doublet may provide a suitable linker.

### Intracellular Domain

The terms “intracellular domain”, “cytoplasmic domain” and “intracellular signaling domain” are used interchangeably herein. The role of the intracellular domain of the CAR is to produce an activation signal to the T cell as soon as the extracellular domain has recognized the antigen.

Examples of intracellular domain sequences that are of particular use in the invention include those derived from an intracellular signaling domain of a lymphocyte receptor chain, a TCR/CD3 complex protein, an Fc receptor subunit, an IL-2 receptor subunit, CD3ζ, FcRγ, FcRβ, CD3γ, CD3δ, CD3ε, CD5, CD22, CD79a, CD79b, CD66d, CD278 (ICOS), FcsRI, DAP10, and DAP12. It is particularly preferred that the intracellular domain in the CAR comprises a cytoplasmic signaling sequence derived from CD3ζ.

The intracellular domain of the CAR can be designed to comprise a signaling domain (such as the CD3ζ signaling domain) by itself or combined with costimulatory domain(s). A costimulatory molecule can be defined as a cell surface molecule that is required for an efficient response of lymphocytes to an antigen. Examples of such molecules include CD27, CD28, 4-1BB (CD137), OX40 (CD134), CD30, CD40, CD244 (2B4), ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. The intracellular signaling portion of the above recited co-stimulatory domains can be used alone or in combination with other co-stimulatory domains. In particular, the CAR can comprise any combination of two or more co-stimulatory domains from the group consisting of CD27, CD28, 4-1BB (CD137), OX40 (CD134), CD30, CD40, CD244 (2B4), ICOS, LFA-1, CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

Thus, for example, the CAR can be designed to comprise a signaling domain such as the CD3ζ signaling domain and two co-stimulatory signaling domains selected from CD28 and CD40, CD28 and 4-1BB (CD137), CD28 and OX40 (CD134), and CD28 and LFA-1.

“First-generation CARs” contain a single signaling domain. CARs containing a signaling domain together with one additional costimulatory domain are termed “second generation” while those containing a signaling domain together with two additional costimulatory domains are listed as “third generation”. For example, first-generation CARs contain solely the CD3ζ chain as a single signaling domain. Second- and third-generation CARs consist of one or two additional costimulatory signaling domains, respectively, such as CD28, CD27, OX-40 (CD134) and 4-1BB (CD137). For example, second-generation CAR may contain CD3ζ and CD28 signaling domains, while third-generation CAR may contain CD3ζ, CD28 and either OX40 (CD134) or 4-1BB (CD137).

The CAR of the invention may be a first generation, a second generation, or a third generation CAR as described hereabove. Preferably, the CAR-T cells is a second or third generation CAR.

“TRUCKs” represent the recently developed ‘fourth-generation’ CARs. TRUCKs (T cells redirected for universal cytokine killing) are CAR-redirected T cells used as vehicles to produce and release a transgenic product that accumulates in the targeted tissue. The product, for example a pro-inflammatory cytokine, may be constitutively produced or induced once the T cell is activated by the CAR. Other substances such as enzymes or immunomodulatory molecules may be produced in the same way and deposited by CAR-redirected T cells in the targeted lesion. This strategy involves two separate transgenes expressing for example (i) the CAR-T cells and (ii) a cell activation responsive promoter linked to a cytokine such as IL-12. Consequently, immune stimulatory cytokine such as IL-12 is secreted upon CAR engagement.

In a particular embodiment, the CAR-T cells is a CAR-T cells of fourth generation as defined above.

### Methods to Obtain a CAR-T Cells

Methods and protocols to obtain CAR-T cells are well known in the art. To obtain CAR-T cells from T cells, transfection, transposon system like the sleeping beauty method or infection thanks to a lentivirus or retroviral vectors can be used (see for example Martinez Marina et al., 2019).

Methods using lentivirus able to transduce T cells to obtain CAR-T cells are well known. For example, and as shown in the present application, a lentivirus stock can be used. Protocols used to obtain CAR-T cells are well known in the art (see for example Okuma Atsushi, 2021. Generation of CAR-T Cells by Lentiviral Transduction).

In this case and according to the invention, the inhibition of RINF and the transformation of the cells in CAR-T cells using a lentivirus can be done using the same lentivirus expressing a shRNA targeting RINF and the CAR construction.

Another method to obtain CAR-T cells from T cells is call sleeping beauty using DNA transposons to transfect the cells (see for example Izsvák et al. 2010).

According to the invention, a retrovirus can be used to generate CAR-T cells.

According to the invention, the CAR-T cells can be CAR-T cells from the first, the second, the third, the fourth or fifth generation.

### TCRs

In a particular embodiment, the immune cell of the invention is a T cells armed with recombinant T Cell Receptor (TCR).

In some embodiments, the genetically engineered antigen receptors include recombinant T cell receptors (TCRs) and/or TCRs cloned from naturally occurring T cells.

A “T cell receptor” or “TCR” refers to a molecule that contains a variable α and β chains (also known as TCRa and TCRp, respectively) or a variable γ and δ chains (also known as TCRy and TCR5, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'IAcad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region. In some embodiments, the TCR chains contain a constant domain. For example, like immunoglobulins, the extracellular portion of TCR chains {e.g., a-chain, β-chain) can contain two immunoglobulin domains, a variable domain {e.g., Va or Vp; typically amino acids 1 to 1 16 based on Kabat numbering Kabat et al., “Sequences of Proteins of Immunological Interest, US Dept. Health and Human Services, Public Health Service National Institutes of Health, 1991, 5th ed.) at the N-terminus, and one constant domain {e.g., a-chain constant domain or Ca, typically amino acids 1 17 to 259 based on Kabat, β-chain constant domain or Cp, typically amino acids 1 17 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD3ζ chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each O′O3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β(or optionally γ and δ) or it may be a single chain TCR construct. In some embodiments, the TCR is a heterodimer containing two separate chains (α and β chains or γ and δ chains) that are linked, such as by a disulfide bond or disulfide bonds.

According to the invention, the antigen binding domain used for the CAR-t cells can be used for the T cells armed with recombinant T Cell Receptor (TCR).

### RINF Inhibition

Inhibition of RINF in the immune cell of the invention (notably T cell or CAR-T cell) can be done by any compound, any agent natural (like RINF inhibitor) or any known method such as genetically method.

Notably, according to the present invention, the immune cell of the invention is genetically modified in order to silence the RINF gene. In particular, the gene coding for RINF is deleted or mutated resulting on a non-viable RNA.

All the techniques known by the skilled man may be used for silencing the RINF gene.

Inhibition of RINF in the immune cell according to the present invention can be permanent and irreversible or transient or reversible. Preferably however, RINF inhibition is permanent and irreversible. Inhibition of RINF in the immune cell of the invention may be achieved prior or after injection of the cell in the targeted patient.

In one embodiment, a RINF inhibitor according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Particular small organic molecules range in size up to about 10000 Da, more particularly up to 5000 Da, more particularly up to 2000 Da and most particularly up to about 1000 Da.

The present invention provides for an isolated single domain antibody, wherein said antibody inhibit RINF.

As used herein the term “single domain antibody” has its general meaning in the art and refers to the single heavy chain variable domain of antibodies of the type that can be found in Camelid mammals which are naturally devoid of light chains. Such single domain antibody is also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus yet another consequence of small size is that a nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in nanobodies being extremely thermostable, stable to extreme pH and to proteolytic digestion, and poorly antigenic. Another consequence is that nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system aminoacid numbering (http://imgt.cines.fr/).

Camel Ig can be modified by genetic engineering to yield a small protein having high affinity for a target, resulting in a low molecular weight antibody-derived protein known as a “nanobody” or “VHH”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antibody fragments are commercially available, for example, from Ablynx, Ghent, Belgium. In certain embodiments herein, the camelid antibody or nanobody is naturally produced in the camelid animal, i.e., is produced by the camelid following immunization with RINF or a peptide fragment thereof, using techniques described herein for other antibodies. Alternatively, the RINF-binding camelid nanobody is engineered, i.e., produced by selection for example from a library of phage displaying appropriately mutagenized camelid nanobody proteins using panning procedures with RINF as a target.

In some embodiments, the single domain antibody is a “humanized” single domain antibody.

As used herein the term “humanized” refers to a single domain antibody of the invention wherein an amino acid sequence that corresponds to the amino acid sequence of a naturally occurring VHH domain has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional chain antibody from a human being. Methods for humanizing single domain antibodies are well known in the art. Typically, the humanizing substitutions should be chosen such that the resulting humanized single domain antibodies still retain the favourable properties of single domain antibodies of the invention. The one skilled in the art is able to determine and select suitable humanizing substitutions or suitable combinations of humanizing substitutions. For example, the single domain antibodies of the invention may be suitably humanized at any framework residue that the single domain antibodies remain soluble and do not significantly loss their affinity for RINF.

In one embodiment, the compound according to the invention is a peptide or a polypeptide.

In a particular embodiment the polypeptide is an antagonist of RINF and is capable to prevent the function of RINF. Particularly, the polypeptide can be a mutated RINF protein or a similar protein without the function of RINF.

In one embodiment, the polypeptide of the invention may be linked to a cell-penetrating peptide” to allow the penetration of the polypeptide in the cell.

The term “cell-penetrating peptides” are well known in the art and refers to cell permeable sequence or membranous penetrating sequence such as penetratin, TAT mitochondrial penetrating sequence and compounds (Bechara and Sagan, 2013; Jones and Sayers, 2012; Khafagy el and Morishita, 2012; Malhi and Murthy, 2012).

The polypeptides of the invention may be produced by any suitable means, as will be apparent to those of skill in the art. In order to produce sufficient amounts of polypeptide or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the RINF inhibitor according to the invention is an inhibitor of RINF gene expression. Particularly, ribozymes, antisense oligonucleotides, siRNAs, miRNA or shRNAs are used for silencing the RINF gene.

Small inhibitory RNAs (siRNAs) can also function as inhibitors of rinf expression for use in the present invention. RINF gene expression can be reduced by contacting a subject or cell with a small double stranded RNA (dsRNA), or a vector or construct causing the production of a small double stranded RNA, such that rinf gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter (or the H1 promoter) to ensure that the shRNA is always expressed. The shRNA hairpin structure is cleaved by the cellular machinery into siRNA, which is then bound to the RNA-induced silencing complex (RISC). In particular SEQ ID NO: 2 may be used. This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene.

Ribozymes can also function as inhibitors of rinf gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of RINF mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of rinf gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, shRNAs, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and particularly cells expressing RINF. Particularly, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a particular type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Particular viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, a specific construct encoding by a vector and containing a shRNA has a sequence a set for in SED ID NO: 2 (see the examples).

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes. For example, a specific expression in Muller glial cells may be obtained through the promoter of the glutamine synthetase gene is suitable. The promoter can also be, e.g., a viral promoter, such as CMV promoter or any synthetic promoters.

In one embodiment, an endonuclease is used for silencing the RINF gene. In one embodiment, the “CRISPR/Cas9” technology is used for silencing the RINF gene.

As used herein, the term “CRISPR” has its general meaning in the art and refers to clustered regularly interspaced short palindromic repeats associated which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or HI-promoted RNA expression vector, although cleavage efficiencies of the artificial sgRNA are lower than those for systems with the crRNA and tracrRNA expressed separately. In some embodiments, the CRISPR-associated endonuclease can be a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microogranisms. Alternatively, the wild type Streptococcus pyrogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of PX330 or PX260 (Addgene, Cambridge, MA). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%), 97%), 98%), or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The Cas9 nuclease sequence can be a mutated sequence. For example the Cas9 nuclease can be mutated in the conserved FiNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks, and the subsequent preferential repair through HDR can potentially decrease the frequency of unwanted indel mutations from off-target double-stranded breaks. The polypeptides that are biologically active variants of a CRISPR-associated endonuclease can be characterized in terms of the extent to which their sequence is similar to or identical to the corresponding wild-type polypeptide. For example, the sequence of a biologically active variant can be at least or about 80% identical to corresponding residues in the wild-type polypeptide. For example, a biologically active variant of a CRISPR-associated endonuclease can have an amino acid sequence with at least or about 80% sequence identity (e.g., at least or about 85%, 90%, 95%, 97%, 98%, or 99% sequence identity) to a CRISPR-associated endonuclease or to a homolog or ortholog thereof. A biologically active variant of a CRISPR-associated endonuclease polypeptide will retain sufficient biological activity to be useful in the present methods. The biologically active variants will retain sufficient activity to function in targeted DNA cleavage. The biological activity can be assessed in ways known to one of ordinary skill in the art and includes, without limitation, in vitro cleavage assays or functional assays.

It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.).

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13). In particular, SEQ ID NO: 3 and SEQ ID NO: 4 may be used.

### Methods of Treatment of the Invention

Immune cells or CAR-T cells obtained by the methods of the invention can be used to improve the immune response and thus can be used to boost the immune system and thus treat diseases like cancer and infectious diseases.

Thus, another aspect of the invention relates to immune cells (or to a population of immune cells of the invention) obtained (or produced) by a method of the invention to improve the immune response.

Particularly, the invention relates to immune cells (or to a population of immune cells of the invention) characterized in that it is defective for RINF for use in a method to improve the immune response.

Particularly, the invention relates to immune cells obtained by a method of the invention for use in the treatment of a cancer or an infectious disease.

Particularly, the invention relates to a method of treating a cancer or an infectious disease in a subject in need thereof, said method comprising administering to the subject a therapeutically effective amount of immune cells of the invention or of a population of immune cells of the invention.

Particularly, the invention relates to immune cells (or of a population of immune cells of the invention) characterized in that it is defective for RINF for use in the treatment of cancer or an infectious disease. Particularly, the immune cells are CAR T cells or T cells armed with recombinant T Cell Receptor (TCR).

In a particular embodiment, the immune cells or the CAR-T cells of the invention can be used in an allogenic treatment.

In a particular embodiment, the immune cells or the CAR-T cells of the invention can be used in inflammatory diseases (auto-inflammatory disease) like lupus, cardiac diseases like cardiac fibrosis, auto-immunes diseases, transplantation or aging related-disorders.

According to the invention, the population of immune cells or of CAR-T cells prepared as described above can be utilized in methods and compositions for adoptive immunotherapy in accordance with known techniques, or variations thereof that will be apparent to those skilled in the art.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (201 1) Nat Rev Clin Oncol. 8(10):577-85). See, e.g., Themeli et al. (2013) Nat Biotechnol. 31 (10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the immune cell or population of immune cells of the invention are derived from a subject, e.g., patient, in need of a treatment and the cells, following isolation and processing are administered to the same subject.

In some embodiments, the cell therapy, e.g., adoptive cell therapy, e.g., adoptive T cell therapy or adoptive CAR-T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

According to the invention, the cancer may be a liquid or a solid cancer.

In one embodiment, the cancer may be a cancer selected from the group consisting in adrenal cortical cancer, anal cancer, bile duct cancer (e.g. perihilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adenocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma,), vaginal cancer, vulvar cancer, and uterine cancer (e.g. uterine leiomyosarcoma).

According to the invention, the infectious diseases can be due to a pathogen like a virus, bacterium, protozoan, prion, viroid, or fungus.

According to the invention, the bacterium can be selected from the group consisting of: Streptococcus pneumoniae; Staphylococcus aureus; Haemophilus influenza, Myoplasma species, Moraxella catarrhalis, Escherichia, e.g., E. coli, Enterobacter, Erwinia, Klebsiella, Proteus, Salmonella, e.g., Salmonella enterica serovar, Typhimurium, Serratia, e.g., Serratia marcescans, and Shigella, as well as Bacilli such as B. subtilis and B. licheniformis, Pseudomonas such as P. aeruginosa, Campylobacter, Mycobacterium tuberculosis, and Streptomyce.

According to the invention, the fungus can be selected from the group consisting of: aspergillus, Candida albicans and Cryptococcus neoformans.

More particularly, the infectious disease is induced by a respiratory virus.

Particularly, the respiratory virus can be Influenza virus, such as the Influenza A virus (IAV) or the Influenza B virus (IAB), adenovirus, metapneumovirus, cytomegalovirus, parainfluenza virus (e.g., hPIV-1, hPIV-2, hPIV-3, hPIV-4), the human rhinovirus (HRV), the Human respiratory syncytial virus (HRSV) or a coronavirus.

As used herein, the term “coronavirus” has its general meaning in the art and refers to any member of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. In particular, the coronavirus particle comprises at least the four canonical structural proteins E (envelope protein), M (membrane protein), N (nucleocapsid protein), and S (spike protein). The S protein is cleaved into 3 chains: Spike protein S1, Spike protein S2 and Spike protein S2′. Production of the replicase proteins is initiated by the translation of ORF1a and ORF1ab via a −1 ribosomal frame-shifting mechanism. This mechanism produces two large viral polyproteins, pp1a and pp1ab, that are further processed by two virally encoded cysteine proteases, the papain-like protease (PLpro) and a 3C-like protease (3CLpro), which is sometimes referred to as main protease (Mpro). Coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), Middle East respiratory syndrome-related coronavirus (MERS-Cov) and SARS-coronavirus (Center for Disease Control), in particular SARS-Cov1 and SARS-Cov2.

According to the invention, the coronavirus can be a MERS-CoV, SARS-CoV, SARS-CoV-2 or any new future family members.

As used herein, the term “treatment” or “treat” refers to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness (e.g., the pattern of dosing used during therapy). A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “subject” denotes a mammal, such as a rodent, a feline, a canine, and a primate. Preferably, a subject according to the invention is a human.

Thus, particularly, the invention also relates to an immune cell obtained by the methods of the invention for use in the treatment of infectious disease induced by a pathogen as described above in a subject in need thereof.

### Therapeutic Composition

In another aspect, the invention relates to a therapeutic composition comprising an immune cell or a population of immune cells obtained by the methods of the invention.

Particularly, the invention relates to a therapeutic composition comprising an immune cell or a population of immune cells according to the invention.

Particularly, the invention relates to a therapeutic composition comprising an immune cell or a population of immune cells obtained by the methods of the invention to improve the immune response.

Particularly, the invention relates to a therapeutic composition comprising an immune cell or a population of immune cells characterized in that it is defective for RINF to improve the immune response.

In another embodiment, the invention relates to a therapeutic composition comprising an immune cell or a population of immune cells obtained by the method of the invention for use in the treatment of cancer and infectious disease.

In another embodiment, the invention relates to a therapeutic composition comprising an immune cell or a population of immune cells characterized in that it is defective for RINF for use in the treatment of cancer and infectious disease.

Particularly, the immune cell is a TCR-transgenic T cells, a modified/engineered T cells or a CAR T cell.

According to the invention, the immune cell or the population of immune cells are administrated in a therapeutically effective amount.

Any therapeutic agent of the invention may be combined with pharmaceutically acceptable excipients, and optionally sustained-release matrices, such as biodegradable polymers, to form therapeutic compositions.

As used herein, the term “therapeutically effective amount” or “effective amount” refers to an amount effective, at dosages and for periods of time necessary, to achieve a desired therapeutic result. A therapeutically effective amount of the immune cell or the population of immune cells of the present invention may vary according to factors such as the disease state, age, sex, and weight of the individual, and the ability of the immune cell or the population of immune cells of the present invention to elicit a desired response in the individual. A therapeutically effective amount is also one in which any toxic or detrimental effects of the immune cell or the population of immune cells of the present invention are outweighed by the therapeutically beneficial effects. The efficient dosages and dosage regimens for the combination of the immune cell or the population of immune cells of the present invention depend on the disease or condition to be treated and may be determined by the persons skilled in the art. A physician having ordinary skill in the art may readily determine and prescribe the effective amount of the pharmaceutical composition required. For example, the physician could start doses of the oligomers of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of the immune cell or the population of immune cells of the invention may, for example, be evaluated in an animal model system predictive of efficacy to treat cancer or infectious disease. Alternatively, this property of a composition may be evaluated by examining the ability of the compound to induce cytotoxicity by in vitro assays known to the skilled practitioner. A therapeutically effective amount of a therapeutic compound may decrease latent reservoirs, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of the immune cell or the population of immune cells of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of the immune cell or the population of immune cells of the present invention is 0.02-100 mg/kg, such as about 0.02-30 mg/kg, such as about 0.05-10 mg/kg or 0.1-3 mg/kg, for example about 0.5-2 mg/kg.

In other words, the quantity of the immune cell or the population of immune cells administered to a subject in need thereof is between 10³ to 10¹⁰ cells per kg. Particularly, the quantity of cells injected is 10⁶ or 10⁷ cells per kg. Particularly the unit to use the immune cell or the population of immune cells of the invention will be most advantageously a number of cells per kg (as shown above).

Administration may be intravenous, intramuscular, intraperitoneal, intratumoral or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans. If desired, an effective daily dose of a pharmaceutical composition may be administered as two, three, four, five, six or more sub-doses administered separately at appropriate intervals throughout the day, optionally, in unit dosage forms. In some embodiments, the oligomers of the present invention are administered by slow continuous infusion over a long period, such as more than 24 hours, in order to minimize any unwanted side effects. An effective dose of the CAR-T cells of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of the CAR-T cells of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In other words, the quantity of the immune cell or the population of immune cells administered to a subject in need thereof is between 10⁴ to 10⁹ cells per kg. Particularly, the quantity of cells injected is 10⁶ or 10⁷ cells per kg. The immune cell or the population of immune cells of the invention can be administrated is 1, 2, 3, 4 or 5 times to the subject in need thereof.

The immune cell or the population of immune cells of the invention may be used alone or in combination with any suitable agent.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Pharmaceutical compositions of the present invention may comprise a further therapeutic active agent. The present invention also relates to a kit comprising an inhibitor according to the invention and a further therapeutic active agent.

For example, anti-cancer agents may be added to the pharmaceutical composition as described below.

Anti-cancer agents may be Melphalan, Vincristine (Oncovin), Cyclophosphamide (Cytoxan), Etoposide (VP-16), Doxorubicin (Adriamycin), Liposomal doxorubicin (Doxil) and Bendamustine (Treanda).

Others anti-cancer agents may be for example cytarabine, anthracyclines, fludarabine, gemcitabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, Epirubicin, 5-fluorouracil, taxanes such as docetaxel and paclitaxel, leucovorin, levamisole, irinotecan, estramustine, etoposide, nitrogen mustards, BCNU, nitrosoureas such as Carmustine and lomustine, vinca alkaloids such as vinblastine, vincristine and vinorelbine, imatinib mesylate, hexamethyhnelamine, topotecan, kinase inhibitors, phosphatase inhibitors, ATPase inhibitors, tyrphostins, protease inhibitors, inhibitors herbimycin A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, MDR inhibitors and Ca2+ ATPase inhibitors.

Additional anti-cancer agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Additional anti-cancer agent may be selected from, but are not limited to, growth or hematopoietic factors such as erythropoietin and thrombopoietin, and growth factor mimetics thereof.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzquinamide, bietanautine, bromopride, buclizine, clebopride, cyclizine, dunenhydrinate, diphenidol, dolasetron, meclizme, methallatal, metopimazine, nabilone, oxypemdyl, pipamazine, scopolamine, sulpiride, tetrahydrocannabinols, thiefhylperazine, thioproperazine and tropisetron. In a preferred embodiment, the antiemetic agent is granisetron or ondansetron.

In another embodiment, the further therapeutic active agent can be a hematopoietic colony stimulating factor. Suitable hematopoietic colony stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-L1 or B7-H1).

Typically, the checkpoint blockade cancer immunotherapy agent is an antibody.

In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The form of the pharmaceutical compositions, the route of administration, the dosage and the regimen naturally depend upon the condition to be treated, the severity of the illness, the age, weight, and sex of the subject, etc.

The pharmaceutical compositions of the invention can be formulated for a topical, oral, intranasal, parenteral, intraocular, intravenous, intramuscular or subcutaneous administration and the like.

Particularly, the pharmaceutical compositions contain vehicles which are pharmaceutically acceptable for a formulation capable of being injected. These may be in isotonic, sterile, saline solutions (monosodium or disodium phosphate, sodium, potassium, calcium or magnesium chloride and the like or mixtures of such salts), or dry, especially freeze-dried compositions. In particular, these may be in organic solvent such as DMSO, ethanol which upon addition, depending on the case, of sterilized water or physiological saline permit the constitution of injectable solutions.

In addition, other pharmaceutically acceptable forms include, e.g. tablets or other solids for oral administration; time release capsules; and any other form currently can be used.

In each of the embodiments of the treatment methods described herein, the CAR-T cells of the invention are delivered in a manner consistent with conventional methodologies associated with management of the disease or disorder for which treatment is sought. In accordance with the disclosure herein, an effective amount of the CAR-T cells of the invention administered to a subject in need of such treatment for a time and under conditions sufficient to prevent or treat the disease or disorder.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention will be further illustrated by the following figures and examples. However, these examples and figures should not be interpreted in any way as limiting the scope of the present invention.

### FIGURES

FIG. 1. The knockdown of RINF leads to an increased number of human T cells generated in vitro. (A) Cell culture growth was monitored for primary T-cells isolated from 6 adult donors transduced with a lentiviral vector expressing either a non-target shRNA (short-hairpin RNA) control sequence (shCtrl) or a shRNA targeting RINF expression (shRINF). Cell growth is indicated in cumulative population doublings. To mimic chronic stimulation, the cells have been stimulated several times with anti-CD3/CD28 (at Day 1, 10, 20 and 30, as indicated by arrows on the time axis). A schematic representation of the lentiviral vector is indicated on the right panel. (B and C) For each donor, the total number of cells generated at the endpoint of the chronic stimulation assay (i.e. at day 20-34, according to the donors) was first determined for both shCtrl and shRINF conditions. Then, the relative fold-increase between the final number of T-cells produced with shRINF versus shCtrl was calculated for each donor. Each dot corresponds to one donor and to the relative fold-increase in the number of cells generated for shRINF compared to shCtrl. Cells were cultured in presence of IL2 (B) or IL7 plus IL15 (C). The number of donors and the median (dashed line) are indicated on the graph. The number of T cells generated is statistically increased in the shRINF condition, compared to shCtrl. *=(p<0.05).

FIG. 2. The knockdown of RINF improves anti-CD19 CAR T cells expansion in vitro. Cell culture growth was monitored for CAR T-cells isolated from 3 adult blood donors. Primary T cells were isolated from fresh adult blood (obtained from Etablissement Frangais du Sang) and transduced first a lentiviral vector expressing an anti-CD19 CAR-T. The following day, another transduction was performed with a lentiviral vector expressing either a non-target shRNA control (shCtrl) or a shRNA targeting RINF expression (shRINF). CAR-T cells were here cultured in presence of IL2 (100 IU/ml). The cells have been stimulated with T Cell TransAct™ (Miltenyi Biotec), a human polyclonal antibody mix of anti-CD3/CD28 (at Day 1, 10, and 20). A schematic representation of the lentiviral vector used to generate the CAR-T (which drives the expression of recombinant human anti-CD19 antibody scFv fragment (Fmc63), is shown on the right panel.

FIG. 3. The knockdown of RINF improves anti-EGFR CAR T cells expansion in vitro. Cell culture growth was monitored for CAR T-cells isolated from 3 adult blood donors. Primary T cells were isolated from fresh adult blood donor (Etablissement Frangais du Sang) and transduced first a lentiviral vector expressing an anti-EGFR CAR (second generation), and one day after with another lentiviral vector expressing either a non-target shRNA control (shCtrl) or a shRNA targeting RINF expression (shRINF). CAR-T cells were here cultured in presence of IL7 and IL15 (10 ng/ml each). The cells have been stimulated with T Cell TransAct™ (Miltenyi Biotec), a human polyclonal antibody mix of anti-CD3/CD28 (at Day 1, 12, 19 28). A schematic representation of the lentiviral vectors used to generate the CAR-T (which drives the expression of recombinant human anti-EGFR antibody scFv fragment (Nimotuzumab)), is shown on the right panel.

FIG. 4. The knockdown of RINF improves CAR T cells persistence and efficacy in vivo. (A) A schematic representation of the experimental design used to assess the CAR T cells functionality in vivo. Briefly, 3 10^{\hat{ }6} A549 cells (a cell line that endogenously expresses the tumor antigen EGFR) were injected subcutaneously in immunocompromised 6- to 8-week-old NSG mice (on day 0). Eleven days later, 1×10⁷ EGFR-CAR T cells were injected intravenously (tail vein). (B) The CAR-T cells number was monitored by flow cytometry in blood (left panel) or, within tumor, after mouse sacrifice (at day 105) and tumor digestion. (C) The tumor burden (left panel) was monitored every 3 to 4 days with an electronic caliper (during approximately 3 months; n=4 mice for PBS, n=6 mice for shCtrl, n=6 mice for shRINF). Tumor volume was calculated by the formula: tumor volume=(length of the tumor)×(width of the tumor)²/2. Tumors were excised and weighted at Day 105, after sacrifice (right panel). CAR T cells (knockdown or not for RINF) were generated from the same blood donor. The number of CAR-T cells was measured by flow cytometry in blood (at day 18, 20, 75, and 97 post-CAR-T injection) or in tumor (after mouse sacrifice and tumor digestion), at 105 days. The tumor weight was also determined after tumor dissection.

FIG. 5. A weak knockdown of RINF (˜20%) was sufficient to improve long-term CAR-T cells efficiency. The relative mRNA expression of RINF was detected by q-RT-PCR (normalized to GAPDH mRNA expression), and expressed in percentage of shRNA controls cells of matched-control donors. For the five donors (19 #, 28 #, 30 #, 31 #, and #36), the observed know-down of RINF (compared to control cells) was ranging from ˜20% to ˜50% at 8-10 days of CAR-T cells ex vivo expansion. Importantly, for Donor #36, the EGFR CAR-T shRINF cells (that presented a RINF knockdown of ˜20%* compared to its control) were used in xenograft model and demonstrated a higher capacity to control tumor growth (this cells are the one used in the experiment presented on FIG. 4C).

FIG. 6. RINF gene disruption by Crispr-Cas9 improves (anti-EGFR and anti-CD19) CAR T cells efficacy in vivo. (A) A schematic representation of the experimental design used to assess the CAR T cells functionality in vivo. Briefly, 4 10^{\hat{ }6} A549/CD19 cells (a cell line that endogenously expresses the tumor antigen EGFR and that ectopically expresses human CD19 (retroviral transduction)), were injected subcutaneously in 6- to 8-week-old NSG mice (21 days before CAR-T infusion). Three weeks after A549 subcutaneous injection, 5×10⁶ EGFR-CAR T cells were injected intravenously (in the tail vein) or ˜1.8×10⁶ CD19-CAR. (B) The tumor burden was monitored one or twice a week with an electronic caliper during approximately ten weeks for anti-EGFR CAR T cells treated mice (left panel, n=6 mice treated with anti-EGFR CAR T/control and n=6 mice treated with anti-EGFR CAR T/RINF KO) and eight weeks for anti-CD19 CAR T cells treated mice (right panel, n=8 mice for control- and n=7 for RINF KO cells). Tumor volume was calculated by the formula: tumor volume=(length of the tumor)×(width of the tumor)²/2. For each CAR-T, Crispr-Cas9 has been used to knock-out RINF gene (white squares) or not (black rounds) before activation.

### EXAMPLE

### Material & Methods

### Cell Lines.

A549, NALM-6, and HEK293T cell lines were obtained from the American Type Culture Collection (ATCC). The A549/CD19 cell line was generated by retroviral transduction and of A549 cells with the addgene vector N^{o}. 127889 allowing the stable ectopic expression of human CD19 has previously described (PMID: 30814732).

### Cell Culture and Activation of Human Primary T-Cells.

Human peripheral blood T lymphocytes (PBT) were purified from blood of healthy donors obtained from Etablissement Frangais du Sang with written informed consent for research use, in accordance with the Declaration of Helsinki. Briefly, Peripheral Blood Mononucleated Cells (PBMC) were separated from fresh blood by Histopaque-1077 (Sigma), according to the manufacturer's instructions. T-cells were thus isolated with the Pan T Cell isolation kit, human (Miltenyi Biotec) and cultured in serum-free TexMACS™ medium with phenol red (Miltenyi Biotec), in presence of 100 U/ml of recombinant human IL-2 (Biolegend) or a combination of human IL-7 and IL-15, at 10 ng/mL each (Miltenyi Biotec). T-cells were seeded at 1 million cells per mL of medium. Cells were cultured at a temperature of 37° C. in a humid atmosphere at 5% C02 saturation. The cells were daily monitored, cultured for up to 34 days post-activation, and kept at a concentration between 1 and 2 million cells/mL. For cell growth monitoring, cell concentration and viability were measured twice a week by using a C-chip disposable Hemocytometer (NanoEntek) by using the trypan blue exclusion method (Life Technologies). Cell proliferation was represented as population doublings (PD) calculated by the formula: PD=Log (N/No)/Log 2, where N is the number of cells counted and No the number of cells seeded at day 0. To mimic a chronic activation, T-cells were activated (at day 0) and re-stimulated every 10 days (i.e. at day 10, 20, and 30) with 5 μl/10⁶ cells with T Cell TransAct™, a human polyclonal antibody mix of anti-CD3/CD28, from Miltenyi Biotec.

### Lentiviral Vectors Driving Short-Hairpin-RNA or Chimeric Antigen Receptors (CAR) Expression.

For short-hairpin-RNA (shRNA)-mediated RINF knockdown experiments, we used the pTRIPDU3/eGFP lentiviral vector (1,2) which drives the short-hairpin-RNA sequences targeting RINF (shRINF) or a non-target sequence control (shCtrl), downstream of the H1 promoter, as previously described (3). The two respective short-hairpin-RNA sequences are presented hereafter:

pTRIPDU3/eGFP/shCtrl (SEQ ID NO: 1):
CCGGCCTAAGGTTAAGTCGCCCTCGTTCAAGAGACGAGG
GCGACTTAACCTTAGGTTTTT
pTRIPDU3/eGFP/shRINF (SEQ ID NO: 2):
CCGGCTTTGATTCTTTCCGACCATTTCAAGAGAATGGTC
GGAAAGAATCAAAGGTTTTTT

Chimeric Antigen Receptors (CAR) constructs were designed by Creative Biolabs. The Lenti-EF1a-ScFv-h(BBζ)-IRES-EGFP-2nd-CAR drives the expression of Recombinant Human Antibody scFv Fragment recognizing human EGFR (Nimotuzumab) or human CD19 target antigens (FMC63).

For some experiments, we used an alternative lentiviral construct enabling to monitor the transduction rate of the cells transduced with both the CAR (mCherry) and/or the shRNA lentiviral vectors (GFP).

### Production of Lentiviral Vectors and T-Cells Transduction.

Production of lentiviral particles were performed by transient co-transfection of HEK293T cells (293LTV cell line, Cell Biolabs) with Fugene HD (Roche) or PEI 40K (Polyethylenimine Linear, MW 40000, Polysciences) with the second-generation packaging system developed by Didier Trono's laboratory (Ecole Polytechnique Fédérale de Lausanne, Switzerland). Briefly, Chimeric Antigen Receptors (CAR) vectors or shRNA-expressing vectors (pTRIPDU3/GFP) were transfected along with the packaging plasmid psPAX2 (Addgene 12260) and the envelope plasmid pMD2.G (Addgene 12259). Viral supernatants were harvested at 48 hours and 72 hours post-transfection, and viral particles were concentrated by ultracentrifugation at 22.000 g for 2 h, at 4° C., and conserved at −80° C. The lentiviral titer was determined 3 days after transduction (based on the GFP-expression rate) and estimated at approximately ˜8. 10⁷ lentiviral particles/mL (for activated primary T-cells). Primary T-cells were activated 24 h before transduction, 10 μl of concentrated lentiviral supernatant were administrated for every 10⁶ primary-T cells in culture. 24 h post-transduction, cells were washed 2 times in PBS 1× (by centrifugation at 300 g) and the cell pellet was resuspended in fresh culture media.

Generation of CAR T Cells Silenced (by a shRNA-Mediated Approach) or Knockout for RINF Gene (by a Crispr-Cas9-Mediated Approach).

Peripheral blood mononuclear cells (PBMCs) were isolated from adult healthy donors' peripheral blood and were cultured in RPMI 1640 supplemented with 10% of inactivated fetal bovine serum (Thermo Fisher Scientific), 2 mM GlutaMAX (Life Technologies) and activated with 5 μl of T Cell TransAct™ per million of cells (Miltenyi Biotec). The following day (Day 1), 10⁶ cells were transduced with 20 μl of CAR lentiviral in presence of 1:200 of total volume Lentiboost (Sirion Biotech). One day following the lentiviral transduction of T-cells with the CAR constructs (i.e. anti-EGFR or anti-CD19), the CAR-T cells were ready for either, (i) a shRNA-mediated RINF silencing, or (ii), a Crispr-Cas9-mediated RINF gene invalidation:

(i) RINF Silencing of CAR-T Cells: The day after lentiviral transduction with the CAR constructs (Day 2), 5 μl of pTRIPDU3/shRNA lentiviral supernatant were added into the medium for 24 hours infection. Cells were washed with PBS 1× and transferred to RPMI 1640 supplemented with 10% FBS, 10 ng/mL IL-7 and 10 ng/ml IL-15 (Miltenyi Biotech) on the following day. Cells expanded for 10 to 12 days were ready for functional experiments.

### (ii) RINF Invalidation by Crispr-Cas9 in CAR-T Cells:

Nucleofection of CAR-T cells was performed on a 4D nucleofector machine (Lonza). Briefly, one day after lentiviral transduction of CAR vectors (Day 2), approximately ˜1.5 million CAR-T cells were electroporated with CRISPR/Cas9 ribonucleoparticles (RNPs) containing 120 pmol of Cas9 protein complexed with 200 pmol of guide RNAs (gRNA) from Thermofisher. To disrupt the coding sequence of CXXC5 gene (5′-GTTGCTTTTGTCCACCGCCA-3′ (SEQ ID NO: 3), and 5′-TGGTGTGTCATCTGCCACTG-3′ (SEQ ID NO: 4)) and compared to a non-target negative control gRNA sequence (TrueGuide sgRNA negative Control, non targeting 1, N^{o} A35526, Thermofisher). After nucleofection, CAR-T were expanded in in RPMI medium containing 10% of FBS (Life Technologies) supplemented with IL-7 and IL-15 (at 10 ng/mL) for ten more days (before infusion in mouse xenograft expanded ex vivo). The percentage of RINF invalidation was estimated by Sanger Sequencing and deconvolution analyses. Briefly, CRISPR/Cas9 edited cells, we proceeded to DNA extraction by (FastPure Blood/Cell/Tissue/Bacterie DNA isolation Mini Kit-BOX2, Vazyme, DC212-02) and PCR amplification of CXXC5 region targeted by our sgRNAs primers. PCR amplicons were sequenced by Sanger sequencing. Deconvolution analysis with DECODR software was performed to determine frequencies of indels causing inactivating frameshift mutations in the target sequence.

### RNA Extraction and Quantitative RT-PCR Analysis.

CAR-T cells expanded for 8-10 days were collected and stored directly at −80° C. for RNA preparation with the TRIzol (Life Technologies) extraction protocol as indicated by the manufacturer's instructions. First-strand cDNA synthesis (reverse transcription) was carried out using a Transcriptor First Strand cDNA Synthesis Kit (cat. n. 489703000, Roche). RINF mRNA expression was quantified by qRT-PCR using SYBRGreen on a Light Cycler 480 machine (Roche) and gene expression was calculated by the 2-ΔΔCT method.

### Mouse Tumor Xenograft Models

6- to 8-week-old NSG mice (Non-Obese Diabetic, SCID gamma mouse, from Charles River laboratories and bred at Cochin Institute) were used to analyze CAR T cells functions in vivo. For A549 model (a cell line that endogenously expresses human EGFR), 3×10⁶ A549 cells were injected subcutaneously on Day 0. Eleven days later, 1×10⁷ EGFR-CAR T cells were injected intravenously. The tumor burden was measured every 3 to 4 days by electronic caliper. Tumor volume was calculated by the formula: tumor volume=(length of the tumor)×(width of the tumor)2/2. For A549/CD19 cells (a cell line that endogenously expresses the tumor antigen EGFR and that ectopically expresses human CD19 (retroviral transduction)), were injected subcutaneously ˜3 weeks before CAR-T intravenous injection.

### Flow Cytometry.

60 μL of blood of NSG mice was collected by retro-orbital sampling method, and then stained with antibodies followed by Red Blood Cell lysis (eBioscience™ 1×RBC Lysis Buffer) and fixed with 2% PFA for 15 minutes on ice. 10 ul of the CountBright absolute counting beads (ThermoFisher Scientific) were added into the blood FACs sample before flow cytometry analysis. The numbers of CAR T cells in the blood were calculated by following formula: CAR T cells number (cells/ul)=(CAR T cell events/beads events)×(beads number/blood volume). Tumors taken from mice were minced with scissors and digested in RPMI 1640 containing 100 μg/ml Dnase I (Roche), 100 μg/ml liberase (Roche) and 500 μg/ml hyaluronidase (Merck) shaking in 37° C. for 30 min, and then milled with 40 μm filter to obtain the single-cell suspension. Afterwards, the cells were washed and stained with LIVE/DEAD Fixable Blue dye (ThermoFisher Scientific) for 20 min followed by antibodies staining for 30 min in the fridge. All samples were fixed with 2% PFA before flow cytometry analysis. Data were acquired by BD Fortessa cytometers and analyzed by FlowJo software (BD Biosciences).

### T-Cells Surface Staining and Cytofluorimetric Analysis.

T-cells were phenotyped at day 30 after first activation. T cells were stained with: LIVE/DEAD™ Fixable Blue Dead Cell Stain (Thermofisher), Brilliant Violet 650™ anti-human CD4 Antibody (clone OKT4 from Biolegend), BUV737 Mouse Anti-Human CD8 Antibody (clone RPA-T8, from BD Optibuild), PerCP/Cyanine5.5 anti-human CD62L Antibody (clone DREG-56, from Biolegend) and Brilliant Violet 711™ anti-human CD45RA Antibody (clone HI100, from Biolegend). First, cells were resuspended in 50 μl of 1:1000 LIVE/DEAD™ Fixable Blue Dead Cell Stain and incubated at 4° C. away from light exposure for 15 minutes. Tubes were thus washed with 1 mL of PBS, then the cells were resuspended in 40 μl of a mix containing all the previously mentioned antibodies at a 1:200 concentration, for 30 minutes. Finally, cells were washed again with PBS then fixed by 15 minutes incubation at 4° C. in PFA 2% and resuspended in 300 μl of PBS for flow cytometry analysis. Cytofluorimetric analysis has been performed on a BD LSRFortessa™ from BD Biosciences. UltraComp eBeads™ Compensation Beads (Thermofisher) have been stained with the different antibodies aforementioned, to acquire a signal to be used as compensation positive control. FlowJo X 10.0.7r2 software have been used to calculate compensation and then analyze FCS data from flow cytometry. Gating has been performed with the help of unstained controls, the same gating has been applied to all conditions in order to allow comparisons among them.

### Statistics.

Significance in population doublings differences have been calculated with paired t-test. Significance in fold increase in cell numbers has been calculated with one sample Wilcoxon test. Correlation among fold change increase at day 34 was assessed by Pearson R calculation.

### Results

### RINF Gene Extinction Leads to an Increased Number of Human T Cells Produced Ex Vivo.

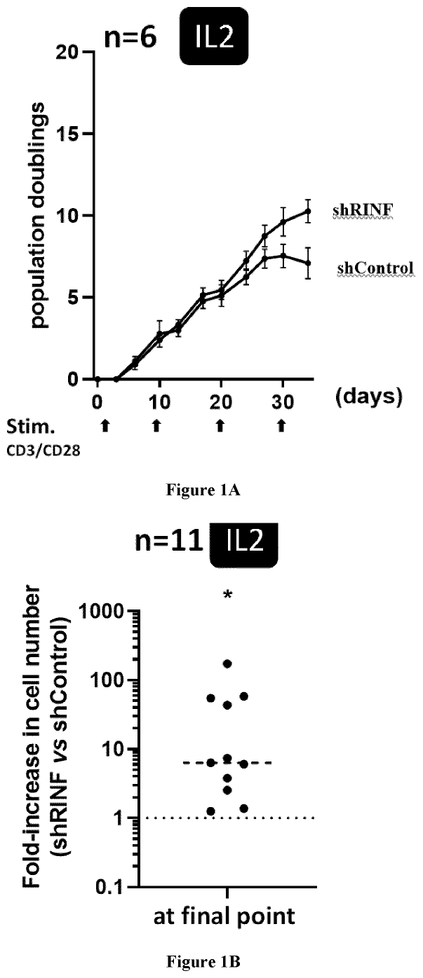

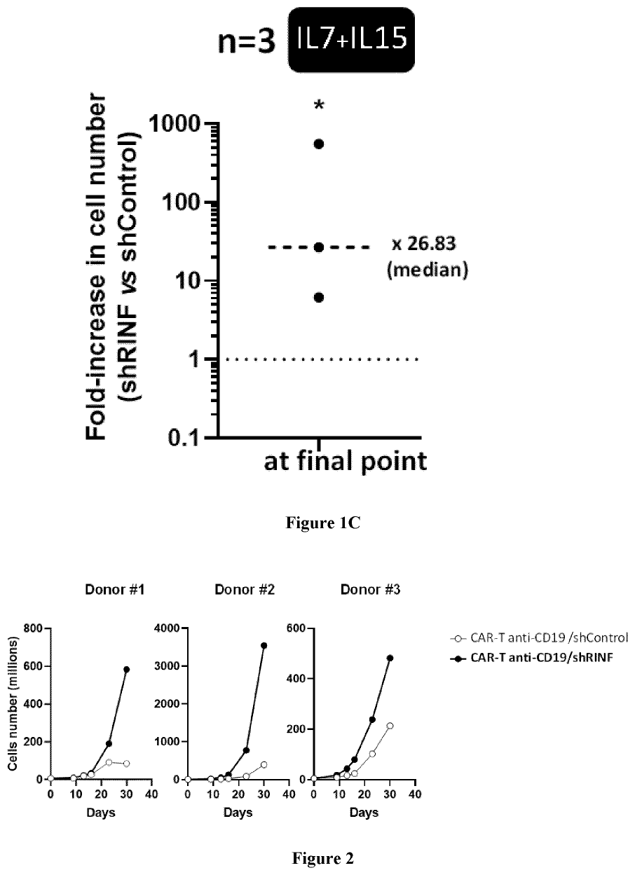

To functionally assess the consequences of RINF knockdown (KD) on human primary T-cells, T-lymphocytes were isolated from Peripheral Blood Mononucleate Cells (PBMC) samples obtained from adult donors. For each donor, two groups of cells were transduced with lentiviral vectors either expressing a non-target shRNA control or a shRNA targeting RINF expression (FIG. 1). These cells underwent to an in vitro chronic stimulation (every 10 days) assay upon TCR and CD28 engagement (i.e. by using an anti-CD3/CD28 antibody mixture), and their expansion was followed by cell counting during approximately 5 weeks. T cells populations exposed to chronic stimulation expanded until reaching a plateau and then started to contract (i.e. become dysfunctional and die). Both shRINF and shCtrl, showed a similar growth kinetic for the first 20 days but after this period of time, a proliferative advantage was observed for T-cells knocked-down (KD) for RINF. Despite the high variability among donors (n=6, FIG. 1A), the number of T cells (here expressed in Population Doublings) became statistically significant from day 30. At the established end point of the experiment (at day 34 after the first stimulation), an almost seven-fold (×6.8) increase was observed in the numbers of T cells in the RINF KD condition, compared with cells transduced with shCtrl lentiviral vector, at least in presence of IL2 (n=11, FIG. 1B). This increase (triggered by the shRNA-mediated RINF silencing) was also noticed in presence of IL7 and IL15 (n=3, FIG. 1C).

### RINF Gene Extinction Improves Anti-CD19 and Anti-EGFR CAR T Cells Expansion Ex Vivo.

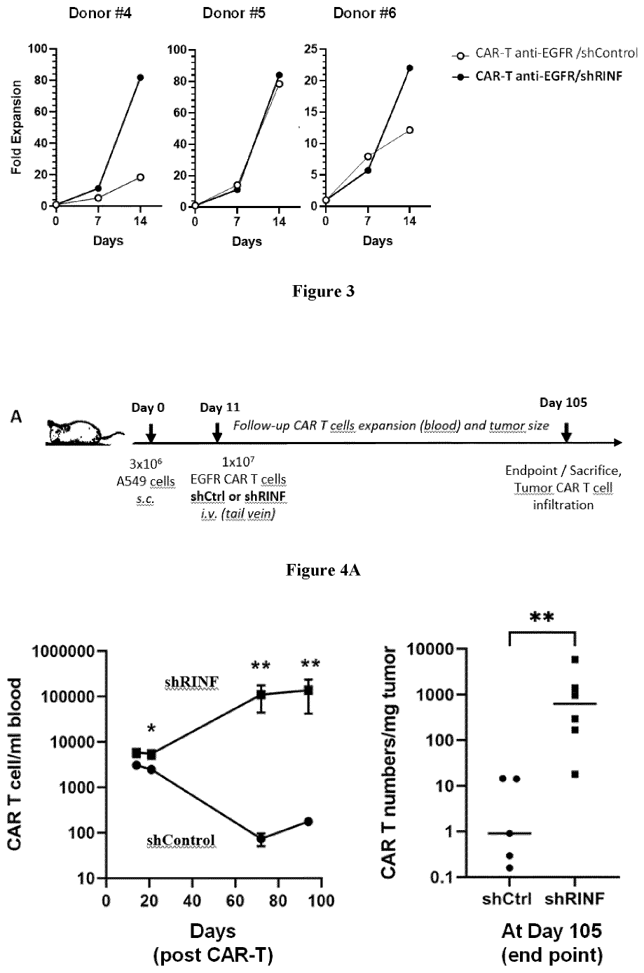

We then wondered if similar results could be observed in T cells genetically engineered to express Chimeric Antigen Receptor (CAR) molecules targeting surface antigens on tumor cells. To test this hypothesis, we first transduced T cells (stimulated at Day 1, and every 10 days with anti-CD3/CD28) with a lentiviral construct driving an anti-CD19 CAR-construct (at Day 0) and then, the following day, with a second lentiviral vector either expressing a non-target shRNA control or a shRNA targeting RINF expression (FIG. 2). After one month of cell expansion (by Day 30), a higher number of CAR-T cells was noticed with the 3 donors tested, indicating than the RINF silencing could increase the number of CAR-T generated ex vivo, at least in presence of IL2. Interestingly, similar data were observed with anti-EGFR CAR-T cells (FIG. 3) that were cultured in the presence of IL7 and IL15, indicating that this biological effect was not restricted to anti-CD19 CAR constructs or to the type of cytokines (IL2 or IL7/IL15) used during the cell culture expansion step. Altogether, these data indicated the RINF knockdown could provide to both T and CAR-T cells an improved capacity of proliferation ex vivo, at least on the long-term.

### The Knockdown of RINF Improves CAR T Cells Persistence and Efficacy In Vivo.

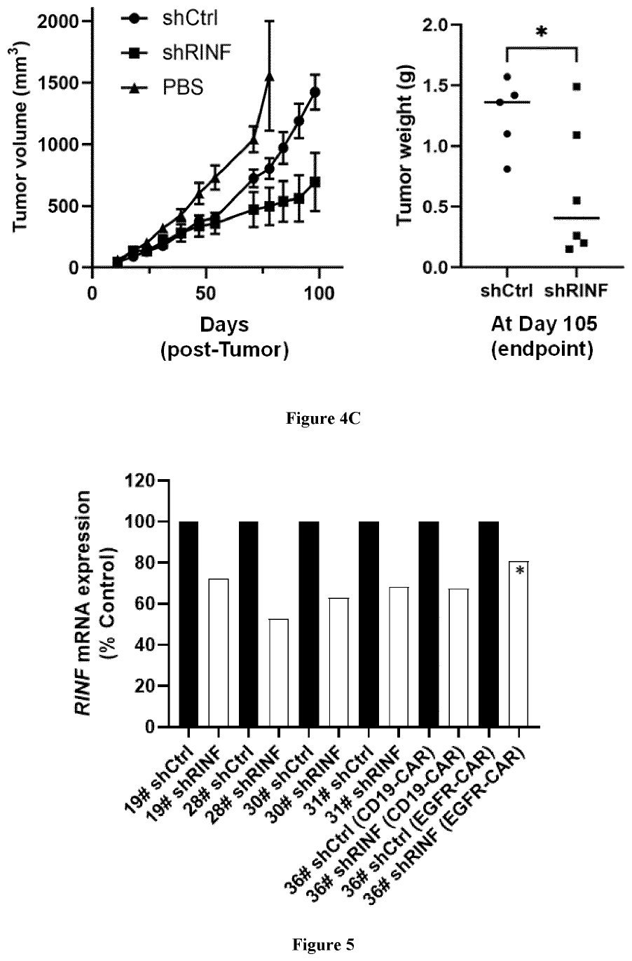

To assess that the CAR-T cells silenced for RINF gene expression are still functional and not altered in their capacity to eradicate tumors, we performed in vivo experiments by using an immunocompromised NSG-mouse model, subcutaneously transplanted with A549 cells, a lung cancer cell line known to endogenously express the EGFR tumor antigen. The design of the experiment is presented on FIG. 4A. Interestingly, even though the same number of CAR-T cells (10 millions) was injected (intravenously in the tail vein) for both conditions (control shRNA or targeting RINF), the number of CAR-T cells transduced with the shRNA-control dropped dramatically few weeks following CAR-T injection (i.e. by 3 weeks post CAR-T injection), while the shRNA-RINF CAR-T cells continued to expand during the following 10 weeks post CAR-T injection (FIG. 4B). These data suggested than RINF targeting could efficiently improve CAR-T cells persistence in vivo. Importantly, at the time of sacrifice (105 days) the average number of CAR-T cells was almost 1000 times higher in the tumors or blood with the shRINF condition, compared to the control condition (FIG. 4B). We also monitored during approximately 3 months, the tumor growth of these mice non-treated with CAR-T cells (mouse PBS control, n=4) or treated with the two types of CAR-T cells, either expressing a shRNA-control (circle, n=5) or a shRNA-targeting RINF (square, n=6) (FIG. 4C). While the shRNA-Control CAR-T cells barely controlled the tumor size of these animals, the tumors of the mouse treated with the shRNA-RINF CAR-T cells were significantly smaller than the one treated with the shRNA-Control CAR-T cells at late stages (after 10 weeks), indicating than the shRINF CAR-T cells were very efficient, even in a solid tumor model (that are known to be refractory to CAR-T cells treatments). This efficacy was confirmed by tumor weighing, that were statistically lighter for RINF knockdown conditions (compared to controls). Altogether, these data suggest that RINF targeting (or knockdown) could improve the in vivo persistence of CAR-T cells, in blood and within the tumors, without compromising their functional efficacy. Interestingly, a partial knockdown of RINF of only ˜20% (compared to the shControl), was apparently sufficient to provide a long-term persistence and improved CAR-T cells efficacy, as observed for Donor #36 (the donor used on FIG. 4C and FIG. 5).

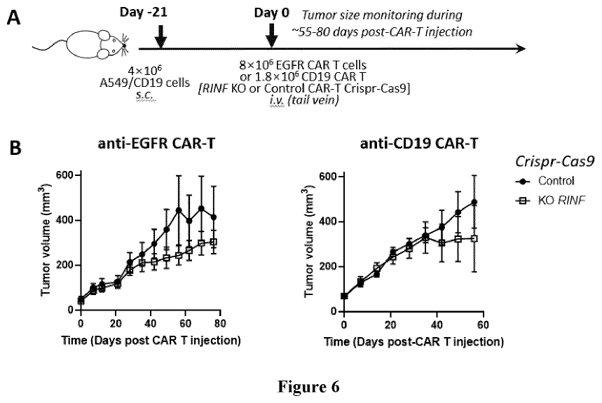

The knockout of RINF gene by CRISPR-Cas9 improves CAR T cells efficacy in vivo. To assess that RINF inhibition would improve CAR-T cells efficacy with another methodological approach than the shRNA-mediated gene silencing, we used the Crispr-Cas9 technology to invalidate RINF in human CAR-T cells (FIG. 6). For the presented experiments, the percentage of RINF invalidation was high and estimated at ˜90%. The CAR-T cells knock-out for RINF gene were first amplified ex vivo during approximately 2 weeks (14 days) before being injected in immunocompromised NSG mice subcutaneously transplanted with A549/CD19 cells (˜4×10 6 A549/CD19 cells, see also experimental design on FIG. 6A). Approximately 3 weeks later, when the tumors were palpable and considered big enough for treatment with CAR-T, approximately ˜5×10 6 anti-EGFR-CAR T cells (left panel) and ˜1.8×10 6 anti-CD19-CAR T cells (right panel) were respectively injected intravenously to each mouse. Thirteen mice were treated with anti-EGFR CAR T cells (left panel), seven of which were treated with CAR-T cells invalidated for RINF gene (white squares) and six were treated with control CAR-T cells (black circles). Twelve mice were treated with anti-CD19 CAR-T cells (right panel), including six for each group of CAR-T cells invalidated or not for RINF. For these experiments, we xenografted A549/CD19 cells, a cell line that express both human CD19 (ectopically) and EGFR (endogenously) antigens, enabling CAR-T cells functional assessment of the anti-EGFR-(FIG. 6B, left panel) or anti-CD19 CAR constructs (FIG. 6B, right panel) by using the same cell line. However, for the types of CAR constructs (anti-EGFR or anti-CD19), CAR-T cells were generated from two distinct donors. The tumor burden was measured once a week by electronic caliper. Tumor volume was calculated by the formula: tumor volume=(length of the tumor)×(width of the tumor)²/2. Interestingly, for the two CAR constructs (anti-CD19 and anti-EGFR), the CAR-T cells Knocked-out for RINF (whites squares) arbored a better efficacy to control tumor growth on the long-term way, than control CAR-T cells.

### REFERENCES

Throughout this application, various references describe the state of the art to which this invention pertains. The disclosures of these references are hereby incorporated by reference into the present disclosure.
1. June C H, Sadelain M. Chimeric Antigen Receptor Therapy. N Engl J Med. 2018 Jul. 5; 379(1):64-73. doi:10.1056/NEJMra1706169. Cited in: Pubmed; PMID 29972754.
2. Gill S, June C H. Going viral: chimeric antigen receptor T-cell therapy for hematological malignancies. Immunol Rev. 2015 January; 263(1):68-89. doi:10.1111/imr.12243. Cited in: Pubmed; PMID 25510272.
3. Newick K, O'Brien S, Moon E, Albelda S M. CAR T Cell Therapy for Solid Tumors. Annu Rev Med. 2017 Jan. 14; 68:139-152. doi:10.1146/annurev-med-062315-120245. Cited in: Pubmed; PMID 27860544.
4. Yong C S M, Dardalhon V, Devaud C, Taylor N, Darcy P K, Kershaw M H. CAR T-cell therapy of solid tumors. Immunol Cell Biol. 2017 April; 95(4):356-363. doi:10.1038/icb.2016.128. Cited in: Pubmed; PMID 28003642.
5. Fraietta J A, Nobles C L, Sammons M A, Lundh S, Carty S A, Reich T J, Cogdill A P, Morrissette J J D, DeNizio J E, Reddy S, Hwang Y, Gohil M, Kulikovskaya I, Nazimuddin F, Gupta M, Chen F, Everett J K, Alexander K A, Lin-Shiao E, Gee M H, Liu X, Young R M, Ambrose D, Wang Y, Xu J, Jordan M S, Marcucci K T, Levine B L, Garcia K C, Zhao Y, Kalos M, Porter D L, Kohli R M, Lacey S F, Berger S L, Bushman F D, June C H, Melenhorst J J. Disruption of TET2 promotes the therapeutic efficacy of CD19-targeted T cells. Nature. 2018 June; 558(7709):307-312. eng. Epub 2018/06/01. doi:10.1038/s41586-018-0178-z. Cited in: Pubmed; PMID 29849141.
6. Ley T J, Ding L, Walter M J, McLellan M D, Lamprecht T, Larson D E, Kandoth C, Payton J E, Baty J, Welch J, Harris C C, Lichti C F, Townsend R R, Fulton R S, Dooling D J, Koboldt D C, Schmidt H, Zhang Q, Osborne J R, Lin L, O'Laughlin M, McMichael J F, Delehaunty K D, McGrath S D, Fulton L A, Magrini V J, Vickery T L, Hundal J, Cook L L, Conyers J J, Swift G W, Reed J P, Alldredge P A, Wylie T, Walker J, Kalicki J, Watson M A, Heath S, Shannon W D, Varghese N, Nagarajan R, Westervelt P, Tomasson M H, Link D C, Graubert T A, DiPersio J F, Mardis E R, Wilson R K. DNMT3A mutations in acute myeloid leukemia. N Engl J Med. 2010 Dec. 16; 363(25):2424-33. doi:10.1056/NEJMoa1005143. Cited in: Pubmed; PMID 21067377.
7. Delhommeau F, Dupont S, Della Valle V, James C, Trannoy S, Masse A, Kosmider O, Le Couedic J P, Robert F, Alberdi A, Lecluse Y, Plo I, Dreyfus F J, Marzac C, Casadevall N, Lacombe C, Romana S P, Dessen P, Soulier J, Viguie F, Fontenay M, Vainchenker W, Bernard O A. Mutation in TET2 in myeloid cancers. N Engl J Med. 2009 May 28; 360(22):2289-301. doi:10.1056/NEJMoa0810069. Cited in: Pubmed; PMID 19474426.
8. Ito K, Lee J, Chrysanthou S, Zhao Y, Josephs K, Sato H, Teruya-Feldstein J, Zheng D, Dawlaty M M, Ito K. Non-catalytic Roles of Tet2 Are Essential to Regulate Hematopoietic Stem and Progenitor Cell Homeostasis. Cell Rep. 2019 Sep. 3; 28(10):2480-2490 e4. doi:10.1016/j.celrep.2019.07.094. Cited in: Pubmed; PMID 31484061.
9. Mayle A, Yang L, Rodriguez B, Zhou T, Chang E, Curry C V, Challen G A, Li W, Wheeler D, Rebel V I, Goodell M A. Dnmt3a loss predisposes murine hematopoietic stem cells to malignant transformation. Blood. 2015 Jan. 22; 125(4):629-38. doi:10.1182/blood-2014-08-594648. Cited in: Pubmed; PMID 25416277.
10. Pendino F, Nguyen E, Jonassen I, Dysvik B, Azouz A, Lanotte M, Segal-Bendirdjian E, Lillehaug J R. Functional involvement of RINF, retinoid-inducible nuclear factor (CXXC5), in normal and tumoral human myelopoiesis. Blood. 2009 Apr. 2; 113(14):3172-81. doi:10.1182/blood-2008-07-170035. Cited in: Pubmed; PMID 19182210.
11. Astori A, Matherat G, Munoz I, Gautier E F, Surdez D, Zermati Y, Verdier F, Zaidi S, Feuillet V, Kadi A, Lauret E, Delattre O, Lefevre C, Fontenay M, Segal-Bendirdjian E, Dusanter-Fourt I, Bouscary D, Hermine O, Mayeux P, Pendino F. The epigenetic regulator RINF (CXXC5) maintains <i>SMAD7</i> expression in human immature erythroid cells and sustains red blood cells expansion. Haematologica. 2020 Nov. 26; Online ahead of print. doi:10.3324/haematol.2020.263558. Cited in: Pubmed; PMID 33241676.
12. Andersson T, Sodersten E, Duckworth J K, Cascante A, Fritz N, Sacchetti P, Cervenka I, Bryja V, Hermanson O. CXXC5 is a novel BMP4-regulated modulator of Wnt signaling in neural stem cells. J Biol Chem. 2009 Feb. 6; 284(6):3672-81. doi:10.1074/jbc.M808119200. Cited in: Pubmed; PMID 19001364.
13. Kim H Y, Yang D H, Shin S W, Kim M Y, Yoon J H, Kim S, Park H C, Kang D W, Min D, Hur M W, Choi K Y. CXXC5 is a transcriptional activator of Flk-1 and mediates bone morphogenic protein-induced endothelial cell differentiation and vessel formation. FASEB J. 2014 February; 28(2):615-26. doi:10.1096/fj.13-236216. Cited in: Pubmed; PMID 24136587.
14. Li G, Ye X, Peng X, Deng Y, Yuan W, Li Y, Mo X, Wang X, Wan Y, Liu X, Chen T, Jiang Z, Fan X, Wu X, Wang Y. CXXC5 regulates differentiation of C2C12 myoblasts into myocytes. J Muscle Res Cell Motil. 2014 December; 35(5-6):259-65. doi:10.1007/s10974-014-9400-2. Cited in: Pubmed; PMID 25433557.
15. Kim M S, Yoon S K, Bollig F, Kitagaki J, Hur W, Whye N J, Wu Y P, Rivera M N, Park J Y, Kim H S, Malik K, Bell D W, Englert C, Perantoni A O, Lee S B. A novel Wilms tumor 1 (WT1) target gene negatively regulates the WNT signaling pathway. J Biol Chem. 2010 May 7; 285(19):14585-93. doi:10.1074/jbc.M109.094334. Cited in: Pubmed; PMID 20220130.
16. Kim H Y, Yoon J Y, Yun J H, Cho K W, Lee S H, Rhee Y M, Jung H S, Lim H J, Lee H, Choi J, Heo J N, Lee W, No K T, Min D, Choi K Y. CXXC5 is a negative-feedback regulator of the Wnt/beta-catenin pathway involved in osteoblast differentiation. Cell Death Differ. 2015 June; 22(6):912-20. doi:10.1038/cdd.2014.238. Cited in: Pubmed; PMID 25633194.
17. Lee S H, Kim M Y, Kim H Y, Lee Y M, Kim H, Nam K A, Roh M R, Min do S, Chung K Y, Choi K Y. The Dishevelled-binding protein CXXC5 negatively regulates cutaneous wound healing. J Exp Med. 2015 Jun. 29; 212(7):1061-80. doi:10.1084/jem.20141601. Cited in: Pubmed; PMID 26056233.
18. Lee S H, Seo S H, Lee D H, Pi L Q, Lee W S, Choi K Y. Targeting of CXXC5 by a Competing Peptide Stimulates Hair Regrowth and Wound-Induced Hair Neogenesis. J Invest Dermatol. 2017 November; 137(11):2260-2269. doi:10.1016/j.jid.2017.04.038. Cited in: Pubmed; PMID 28595998.
19. Ma S, Wan X, Deng Z, Shi L, Hao C, Zhou Z, Zhou C, Fang Y, Liu J, Yang J, Chen X, Li T, Zang A, Yin S, Li B, Plumas J, Chaperot L, Zhang X, Xu G, Jiang L, Shen N, Xiong S, Gao X, Zhang Y, Xiao H. Epigenetic regulator CXXC5 recruits DNA demethylase Tet2 to regulate TLR7/9-elicited IFN response in pDCs. J Exp Med. 2017 May 1; 214(5):1471-1491. doi:10.1084/jem.20161149. Cited in: Pubmed; PMID 28416650.
20. Treppendahl M B, Mollgard L, Hellstrom-Lindberg E, Cloos P, Gronbaek K.

Downregulation but lack of promoter hypermethylation or somatic mutations of the potential tumor suppressor CXXC5 in MDS and AML with deletion 5q. Eur J Haematol. 2013 March; 90(3):259-60. doi:10.1111/ejh.12045. Cited in: Pubmed; PMID 23190153.
21. Gelsi-Boyer V, Trouplin V, Adelaide J, Bonansea J, Cervera N, Carbuccia N, Lagarde A, Prebet T, Nezri M, Sainty D, Olschwang S, Xerri L, Chaffanet M, Mozziconacci M J, Vey N, Birnbaum D. Mutations of polycomb-associated gene ASXL1 in myelodysplastic syndromes and chronic myelomonocytic leukaemia. Br J Haematol. 2009 June; 145(6):788-800. doi:10.1111/j.1365-2141.2009.07697.x. Cited in: Pubmed; PMID 19388938.
22. Knappskog S, Myklebust L M, Busch C, Aloysius T, Varhaug J E, Lonning P E, Lillehaug J R, Pendino F. RINF (CXXC5) is overexpressed in solid tumors and is an unfavorable prognostic factor in breast cancer. Ann Oncol. 2011 October; 22(10):2208-15. doi:10.1093/annonc/mdq737. Cited in: Pubmed; PMID 21325450.
23. Yasar P, Ayaz G, Muyan M. Estradiol-Estrogen Receptor alpha Mediates the Expression of the CXXC5 Gene through the Estrogen Response Element-Dependent Signaling Pathway. Sci Rep. 2016 Nov. 25; 6:37808. doi:10.1038/srep37808. Cited in: Pubmed; PMID 27886276.
24. Fang L, Wang Y, Gao Y, Chen X. Overexpression of CXXC5 is a strong poor prognostic factor in ER+ breast cancer. Oncol Lett. 2018 July; 16(1):395-401. doi:10.3892/ol.2018.8647. Cited in: Pubmed; PMID 29928427.
25. Astori A, Fredly H, Aloysius T A, Bullinger L, Mansat-De Mas V, de la Grange P, Delhommeau F, Hagen K M, Recher C, Dusanter-Fourt I, Knappskog S, Lillehaug J R, Pendino F, Bruserud O. CXXC5 (retinoid-inducible nuclear factor, RINF) is a potential therapeutic target in high-risk human acute myeloid leukemia. Oncotarget. 2013 September; 4(9):1438-48. Cited in: Pubmed; PMID 23988457.
26. Bruserud O, Reikvam H, Fredly H, Skavland J, Hagen K M, van Hoang T T, Brenner A K, Kadi A, Astori A, Gjertsen B T, Pendino F. Expression of the potential therapeutic target CXXC5 in primary acute myeloid leukemia cells—high expression is associated with adverse prognosis as well as altered intracellular signaling and transcriptional regulation. Oncotarget. 2015 Feb. 20; 6(5):2794-811. Cited in: Pubmed; PMID 25605239.
27. Kuhnl A, Valk P J, Sanders M A, Ivey A, Hills R K, Mills K I, Gale R E, Kaiser M F, Dillon R, Joannides M, Gilkes A, Haferlach T, Schnittger S, Duprez E, Linch D C, Delwel R, Lowenberg B, Baldus C D, Solomon E, Burnett A K, Grimwade D. Downregulation of the Wnt inhibitor CXXC5 predicts a better prognosis in acute myeloid leukemia. Blood. 2015 May 7; 125(19):2985-94. doi:10.1182/blood-2014-12-613703. Cited in: Pubmed; PMID 25805812.
28. He Y, Wei T, Ye Z, Orme J J, Lin D, Sheng H, Fazli L, Jeffrey Karnes R, Jimenez R, Wang L, Wang L, Gleave M E, Wang Y, Shi L, Huang H. A noncanonical A R addiction drives enzalutamide resistance in prostate cancer. Nat Commun. 2021 Mar. 9; 12(1):1521. doi:10.1038/s41467-021-21860-7. Cited in: Pubmed; PMID 33750801.
29. Kim M Y, Kim H Y, Hong J, Kim D, Lee H, Cheong E, Lee Y, Roth J, Kim D G, Min do S, Choi K Y. CXXC5 plays a role as a transcription activator for myelin genes on oligodendrocyte differentiation. Glia. 2016 March; 64(3):350-62. doi:10.1002/glia.22932. Cited in: Pubmed; PMID 26462610.
30. Lee I, Choi S, Yun J H, Seo S H, Choi S, Choi K Y, Lee W. Crystal structure of the PDZ domain of mouse Dishevelled 1 and its interaction with CXXC5. Biochem Biophys Res Commun. 2017 Apr. 8; 485(3):584-590. doi:10.1016/j.bbrc.2016.12.023. Cited in: Pubmed; PMID 27932247.
31. Zhang M, Wang R, Wang Y, Diao F, Lu F, Gao D, Chen D, Zhai Z, Shu H. The CXXC finger 5 protein is required for DNA damage-induced p53 activation. Sci China C Life Sci. 2009 June; 52(6):528-38. doi:10.1007/s11427-009-0083-7. Cited in: Pubmed; PMID 19557330.
32. Ayaz G, Razizadeh N, Yasar P, Kars G, Kahraman D C, Saatci O, Sahin O, Cetin-Atalay R, Muyan M. CXXC5 as an unmethylated CpG dinucleotide binding protein contributes to estrogen-mediated cellular proliferation. Sci Rep. 2020 Apr. 6; 10(1):5971. doi:10.1038/s41598-020-62912-0. Cited in: Pubmed; PMID 32249801.
33. Ko M, An J, Bandukwala H S, Chavez L, Aijo T, Pastor W A, Segal M F, Li H, Koh K P, Lahdesmaki H, Hogan P G, Aravind L, Rao A. Modulation of TET2 expression and 5-methylcytosine oxidation by the CXXC domain protein IDAX. Nature. 2013 May 2; 497(7447):122-6. doi:10.1038/nature12052. Cited in: Pubmed; PMID 23563267.
34. Ravichandran M, Lei R, Tang Q, Zhao Y, Lee J, Ma L, Chrysanthou S, Lorton B M, Cvekl A, Shechter D, Zheng D, Dawlaty M M. Rinf Regulates Pluripotency Network Genes and Tet Enzymes in Embryonic Stem Cells. Cell Rep. 2019 Aug. 20; 28(8):1993-2003 e5. doi:10.1016/j.celrep.2019.07.080. Cited in: Pubmed; PMID 31433977.
35. Aras S, Pak O, Sommer N, Finley R, Jr., Huttemann M, Weissmann N, Grossman L I. Oxygen-dependent expression of cytochrome c oxidase subunit 4-2 gene expression is mediated by transcription factors RBPJ, CXXC5 and CHCHD2. Nucleic Acids Res. 2013 Feb. 1; 41(4):2255-66. doi:10.1093/nar/gks1454. Cited in: Pubmed; PMID 23303788.
36. Tsuchiya Y, Naito T, Tenno M, Maruyama M, Koseki H, Taniuchi I, Naoe Y. ThPOK represses CXXC5, which induces methylation of histone H3 lysine 9 in Cd40lg promoter by association with SUV39H1: implications in repression of CD40L expression in CD8+ cytotoxic T cells. J Leukoc Biol. 2016 Feb. 19. doi:10.1189/jlb.1A0915-396RR. Cited in: Pubmed; PMID 26896487.
37. Marshall P A, Hernandez Z, Kaneko I, Widener T, Tabacaru C, Aguayo I, Jurutka P W. Discovery of novel vitamin D receptor interacting proteins that modulate 1,25-dihydroxyvitamin D3 signaling. J Steroid Biochem Mol Biol. 2012 October; 132(1-2):147-59. doi:10.1016/j.jsbmb.2012.05.001. Cited in: Pubmed; PMID 22626544.
38. L'Hote D, Georges A, Todeschini A L, Kim J H, Benayoun B A, Bae J, Veitia R A. Discovery of novel protein partners of the transcription factor FOXL2 provides insights into its physiopathological roles. Hum Mol Genet. 2012 Jul. 15; 21(14):3264-74. doi:10.1093/hmg/dds170. Cited in: Pubmed; PMID 22544055.
39. Wang X, Liao P, Fan X, Wan Y, Wang Y, Li Y, Jiang Z, Ye X, Mo X, Ocorr K, Deng Y, Wu X, Yuan W. CXXC5 Associates with Smads to Mediate TNF-alpha Induced Apoptosis. Curr Mol Med. 2013 September; 13(8):1385-96. Cited in: Pubmed; PMID 23906331.
40. Mackensen A, Muller F, Mougiakakos D, Boltz S, Wilhelm A, Aigner M, Volkl S, Simon D, Kleyer A, Munoz L, Kretschmann S, Kharboutli S, Gary R, Reimann H, Rosler W, Uderhardt S, Bang H, Herrmann M, Ekici A B, Buettner C, Habenicht K M, Winkler T H, Kronke G, Schett G. Anti-CD19 CAR T cell therapy for refractory systemic lupus erythematosus. Nat Med. 2022 Sep. 15. doi:10.1038/s41591-022-02017-5. Cited in: Pubmed; PMID 36109639.
41. Aghajanian H, Kimura T, Rurik J G, Hancock A S, Leibowitz M S, Li L, Scholler J, Monslow J, Lo A, Han W, Wang T, Bedi K, Morley M P, Linares Saldana R A, Bolar N A, McDaid K, Assenmacher C A, Smith C L, Wirth D, June C H, Margulies K B, Jain R, Pure E, Albelda S M, Epstein J A. Targeting cardiac fibrosis with engineered T cells. Nature. 2019 September; 573(7774):430-433. Epub 20190911. doi:10.1038/s41586-019-1546-z. Cited in: Pubmed; PMID 31511695.
42. Amor C, Feucht J, Leibold J, Ho Y J, Zhu C, Alonso-Curbelo D, Mansilla-Soto J, Boyer J A, Li X, Giavridis T, Kulick A, Houlihan S, Peerschke E, Friedman S L, Ponomarev V, Piersigilli A, Sadelain M, Lowe S W. Senolytic CAR T cells reverse senescence-associated pathologies. Nature. 2020 July; 583(7814):127-132. doi:10.1038/s41586-020-2403-9. Cited in: Pubmed; PMID 32555459.
43. Amsellem S, Ravet E, Fichelson S, Pflumio F, Dubart-Kupperschmitt A. Maximal lentivirus-mediated gene transfer and sustained transgene expression in human hematopoietic primitive cells and their progeny. Mol Ther. 2002 November; 6(5):673-7. Cited in: Pubmed; PMID 12436963.
44. Sirven A, Ravet E, Charneau P, Zennou V, Coulombel L, Guetard D, Pflumio F, Dubart-Kupperschmitt A. Enhanced transgene expression in cord blood CD34(+)-derived hematopoietic cells, including developing T cells and NOD/SCID mouse repopulating cells, following transduction with modified trip lentiviral vectors. Mol Ther. 2001 April; 3(4):438-48. doi:10.1006/mthe.2001.0282. Cited in: Pubmed; PMID 11319904.

1. An immune cell characterized in that it is defective for Retinoid-Inducible Nuclear Factor (RINF).

2. The immune cell according to claim 1 wherein a gene coding for RINF is deleted or wherein the gene coding for RINF is mutated resulting in a non-viable RNA.

3. The immune cell according to claim 1, wherein the immune cell is a lymphocyte.

4. The immune cell according to claim 3 wherein the T cell is a CAR-T cell or a T cell armed with a recombinant T Cell Receptor (TCR).

5. A population of immune cells according to claim 1.

6. An ex vivo or in vitro method to obtain improved immune cells that are defective for RINF, comprising:
i. isolating immune cells from a sample obtained from a subject;
ii. inhibiting expression and/or activity of RINF in the immune cells.

7. An ex vivo or in vitro method to obtain CAR-T cells that are defective for RINF comprising the following steps:
i. isolating an T cells from a sample obtained from a subject;
ii. transforming the T cells into CAR-T cells;
iii. inhibiting expression and/or activity of RINF in the CAR-T cells obtained in step ii).

8. The ex vivo or in vitro method according to claim 6, wherein the inhibition of RINF is performed using a ribozyme, an antisense oligonucleotide, a siRNA, miRNA or shRNAs.

9. (canceled)

10. (canceled)

11. A method of treating a cancer or an infectious disease in a subject in need thereof, said method comprising administering to the subject a therapeutically effective amount of immune cells or of a population of immune cells according to claim 1.

12. A therapeutic composition comprising an immune cell or a population of immune cells according to claim 1.

13. The immune cell according to claim 3, wherein the lymphocyte is a T cell, a B cell or an NK cell.


SEQUENCE LISTING (4 sequences)

SEQ ID NO: 1 - DNA, 60 bp, synthetic construct
  source 1..60: mol_type = other DNA
ccggcctaaggttaagtcgccctcgttcaagagacgagggcgacttaaccttaggttttt

SEQ ID NO: 2 - DNA, 60 bp, synthetic construct
  source 1..60: mol_type = other DNA
ccggctttgattctttccgaccatttcaagagaatggtcggaaagaatcaaaggtttttt

SEQ ID NO: 3 - RNA, 20 nt, synthetic construct
  source 1..20: mol_type = other RNA
gttgcttttgtccaccgcca

SEQ ID NO: 4 - RNA, 20 nt, synthetic construct
  source 1..20: mol_type = other RNA
tggtgtgtcatctgccactg